(19) United States
(12) Patent Application Publication
(10) Pub. No.: US 2008/0194900 A1
(43) Pub. Date: Aug. 14, 2008

(54) Steam Cracking with Naphtha Dearomatization

(76) Inventor: Vasant L. Bhirud, Katy, TX (US)

Correspondence Address: LUNDEEN & LUNDEEN, PLLC, PO BOX 131144, HOUSTON, TX, 77219-1144, US

(21) Appl. No.: 11/576,410

(22) PCT Filed: Dec. 9, 2005
(86) PCT No.: PCT/US2005/044545

§ 371 Date: Mar. 30, 2007

Related U.S. Application Data
(60) Provisional application No. 60/634,956, filed on Dec. 10, 2004.

(51) Int. Cl. C07C 4/02

(52) U.S. Cl. 585/648

(57) ABSTRACT

Disclosed is a process for upgrading a naphtha feed stream comprising light naphtha, heavy naphtha, or a combination thereof, for supplying to a cracking process. A naphtha feed stream can be supplied to a hydrotreater 142 to remove impurities, followed by dearomatization in an aromatics extraction unit 136. A dearomatized naphtha stream 104 can be supplied to a cracking process 112 and a recovery process 116 to produce various streams including ethylene 122 and propylene 124 for collection, ethane 110 and propane 108 for recycle to the cracking process 112, and a pyrolysis gas stream 128 which can be further treated to produce a C5 olefins stream 106, a C6-C8 stream 104, a C9+ stream 134, and a fuel oil stream 140.

### BACKGROUND OF THE INVENTION

This invention relates to a process for steam cracking of grade and/or off-grade naphtha, and more particularly to steam of catalytic cracking with dearomatization of the naphtha feed for the production of ethylene and propylene.

Approximately half of the world's ethylene capacity is produced by the steam cracking of naphtha feed streams. For purposes of this application, naphtha has a boiling range from C5 to 200° C., and is generally produced by the fractionation of crude oil. Naphtha can comprise light and heavy naphtha. Light naphtha is typically characterized by a boiling point of less than 100° C. and heavy naphtha is typically characterized by a boiling point of between 100° and 200° C. Generally, heavy naphtha has a lower paraffin and higher aromatics content than light naphtha, making it less suitable as feedstock in the production of ethylene without upgrading. Naphtha steam cracking suitability is determined by the composition of paraffins, olefins, naphthenes and aromatics, each of which can be used to produce ethylene and propylene. Aromatics are generally not a desired feed component in the production of ethylene and propylene, and therefore the aromatics content of the naphtha feed can play an important role in determining suitability for cracking. During steam cracking, the aromatic compounds typically produce undesirable fuel oil. Polymers synthesized from aromatic compounds are often responsible for quench oil tower fouling, which can result in unscheduled shutdowns of the steam crackers.

Naphtha streams rich in paraffins and low in aromatics are generally preferred for steam cracking. For example, in Eastern Asia, the design and operation of crackers requires a minimum paraffin content of approximately 65% by weight, typically specified as open spec naphtha (OSN). Most Middle East naphtha feedstock complies with the Eastern Asia OSN specifications. However, much of the non-Middle East sourced naphtha feeds are poor in paraffins and rich in aromatics, resulting in compositions which do not comply with the OSN specifications and therefore are not useful as steam cracker feeds in the prior art steam cracking processes.

Heavy naphthas recovered around the world can vary greatly in the amount of total paraffins and aromatics. The paraffins content can range between approximately 27% and 70% by volume, the naphthenes content between approximately 15% and 60% by volume, and the aromatics content between 10 and 36% by volume. One example of grade naphtha is Basrah Heavy naphtha produced in Iraq, having a boiling point range between 65° and 175° C., a total paraffin content of approximately 69% by volume, a naphthene content of approximately 21% by volume, and an aromatics content of approximately 10% by volume. In comparison, an example of off-grade naphtha is Mubarak crude produced in the United Arab Emirates, having a boiling point range between 104° and 182° C., a total paraffin content of approximately 50% by volume, a naphthenes content of approximately 30% by volume, and an aromatics content of approximately 20% by volume.

For purposes of this application, naphtha which meets OSN specification will be termed “grade” naphtha, while naphtha not meeting OSN specifications (typically a naphtha feed rich in aromatics and/or paraffin poor) will be termed here as “off grade” naphtha.

In U.S. Pat. No. 6,210,561, Bradow et al. disclose a method for steam cracking a hydrocarbon feed wherein the hydrocarbon feed is treated in a hydrotreating zone to remove nitrogen and sulfur compounds. The hydrotreated stream is then supplied to an aromatics saturation zone prior to cracling of the hydrocarbon effluent.

In U.S. Pat. No. 6,149,800, laccino et al. disclose a method for increasing olefin yields from heavy hydrocarbon feedstock. The process comprises hydroprocessing a feedstock in the boiling range of distillate and above, wherein the feedstock and hydrogen treat gas flow countercurrent to one another.

Patents of note include U.S. Pat. Nos. 4,647,368; 4,927,525; 5,053,579; 5,292,976; 5,396,010; 5,414,172; 5,643,441; 5,685,972; and 5,865,988, disclosing naphtha upgrading; U.S. Pat. Nos. 4,877,581 and 4,839,023, disclosing gas oil upgrading; U.S. Pat. Nos. 5,045,174; 5,906,728; and 6,149,800, disclosing the upgrading of stream cracker feeds; and 6,210,561; 6,407,301; and 6,441,263, each of which is herein incorporated by reference.

The present invention also provides a process whereby off-grade naphtha streams can be upgraded for use as steam cracker feedstock by removing the aromatics from the off-grade naphtha stream. By removing the aromatics from some naphtha feeds, the paraffin content can be increased to at least 65%, thereby meeting the OSN specifications. In some cases dearomatized naphtha may be suitable for steam cracking even though it may not meet OSN specifications.

### SUMMARY OF THE INVENTION

The present invention provides a naphtha cracking method where the feedstock can include an off-grade naphtha stream. The naphtha feedstock can be dearomatized for feed to the cracking process. The naphtha feedstock dearomatization can be conveniently integrated with aromatics extraction from the cracker effluent to increase aromatics production.

In one embodiment of the present invention, an olefins process for steam cracking naphtha is provided. The process includes: (a) recovering olefins and pyrolysis gasoline streams from a steam cracking furnace effluent, (b) hydrogenating the pyrolysis gasoline stream and recovering a C₆-C₈ stream therefrom, (c) hydrotreating an aromatics-containing naphtha stream to obtain a naphtha feed stream lean in nitrogen, sulfur, arsenic, lead, or a combination thereof, (d) dearomatizing the C₆-C₈ stream with the naphtha feed stream in a common aromatics extraction unit to obtain a raffinate stream, and (e) feeding the raffinate stream to the steam cracking furnace.

The aromatics-containing naphtha stream can comprise a paraffins content of less than 65 weight percent. The aromatics-containing naphtha stream can comprise an aromatics content of 10 weight percent or more. The steam cracking furnace effluent can comprise a propylene to ethylene weight ratio from 0.3 to 0.8, i.e. the same cracker severity as grade naphtha feed, or in another embodiment, a propylene to ethylene weight ratio from 0.4 to 0.6. The process can further comprise feeding a second naphtha stream to the steam cracking furnace, wherein the second naphtha stream comprises 65 weight percent or more paraffins and no more than 10 weight percent aromatics. The pyrolysis gasoline can be hydrogenated using commercial hydrogenation processes, such as for example, those offered by IFP, UOP, BASF, and others The fouling in a quench oil tower receiving the steam furnace cracking effluent can be inhibited. The process can further comprise recovering ethane and propane from the steam cracking furnace effluent and recycling the recovered ethane and propane to the steam cracking furnace. The process can further comprise recovering a C5 olefins stream from the pyrolysis gasoline hydrogenation and recycling the C5 olefins stream to the steam cracking furnace. The process can further comprise hydrotreating a second naphtha stream, wherein the second naphtha stream comprises 65 weight percent or more paraffins and no greater than 10 weight percent aromatics. The aromatics containing naphtha stream can comprise heavy naphtha.

In another embodiment, the invention provides an olefins process for steam cracking a naphtha stream comprising aromatics. The process includes the steps of: (a) recovering olefins and pyrolysis gasoline streams from a steam cracking furnace effluent, (b) hydrogenating the pyrolysis gasoline stream and recovering a C6-C8 stream therefrom, (c) hydrotreating a heavy naphtha stream comprising aromatics to obtain a heavy naphtha stream lean in nitrogen, sulfur, arsenic, lead, or a combination thereof, (d) reforming the hydrotreated heavy naphtha stream in a catalytic reformer to obtain a reformate comprising aromatics, (e) dearomatizing the C6-C8 stream with the reformate in a common aromatics unit to obtain a mixed stream comprising C6-C8 raffinate, reformate raffinate, and a dearomatized heavy naphtha stream, and (e) feeding the mixed stream to the steam cracking furnace.

The process can further include hydrotreating a second aromatics-containing heavy naphtha stream in a second hydrotreater to obtain a second hydrotreated heavy naphtha stream lean in nitrogen, sulfur, arsenic, lead, or a combination thereof; and dearomatizing the heavy naphtha stream, the reformate and the C6-C8 stream in the common aromatics extraction unit. The process can further include supplying a portion of the hydrotreated heavy naphtha stream to the reformer, and dearomatizing the balance of the hydrotreated heavy naphtha stream with the C6-C8 stream and the reformate raffinate. The process can further include reforming a hydrocracker naphtha stream with a portion of the hydrotreated heavy naphtha in the catalytic reformer to obtain a reformate stream.

In another embodiment of the present invention, an olefins process unit for steam cracking an aromatics-containing naphtha stream is provided. The process unit includes: (a) one or more steam cracking furnaces to produce a pyrolysis effluent, (b) a recovery unit to recover olefins and pyrolysis gasoline streams from the pyrolysis effluent, (c) a gasoline hydrogenation unit to hydrogenate the pyrolysis gasoline stream and recover a C₆-C₈ stream, (d) a hydrotreating unit to remove nitrogen, sulfur, arsenic, lead, or a combination thereof from an aromatics-containing naphtha stream to obtain a naphtha feed stream, (e) a common aromatics extraction unit to dearomatize the C₆-C₈ stream together with the naphtha feed stream to obtain a raffinate stream, and (f) a line to feed the raffinate stream with dearomatized naphtha to the steam cracking furnace.

The olefins process unit can further comprise lines to recycle ethane and propane streams from the recovery unit to the steam cracking furnace. The olefins process unit can further comprise a line to recycle a C₅ olefins stream from the gasoline hydrogenation unit to the steam cracking furnace.

### BRIEF DESCRIPTION OF THE DRAWINGS

For a more detailed description of the illustrated embodiments of the present invention, reference will now be made to the accompanying drawings, wherein:

FIG. 1 is a block flow diagram of a prior art naphtha based steam cracker having an aromatics extraction unit for treating the furnace effluent.

FIG. 2 is a block flow diagram of a naphtha based steam cracking unit having an integrated aromatics extraction unit wherein furnace feed comprising off-grade naphtha can be supplied to an aromatics extraction unit.

FIG. 3 is a block flow diagram of a naphtha based steam cracking unit having an integrated aromatics extraction unit wherein a portion of the furnace feed is an off-grade naphtha supplied to an aromatics extraction unit.

FIG. 4 is a block flow diagram of a naphtha based steam cracking unit having an integrated aromatics extraction unit wherein a portion of the naphtha stream is supplied to a dearomatizer.

FIG. 5 is a block flow diagram of a naphtha based steam cracking unit having an integrated hydrotreater and dearomatization unit for the removal of aromatics from a heavy naphtha feed.

FIG. 6 is a block flow diagram of a naphtha based steam cracking unit having an aromatics removal unit, wherein a heavy naphtha feed is supplied to a catalytic reformer upstream from the aromatics extraction unit.

FIG. 7 is a block flow diagram of a naphtha based steam cracking unit having an integrated aromatics removal unit, wherein a portion of a heavy naphtha feed is supplied to a catalytic reformer upstream from the aromatics extraction unit, and a portion of the heavy naphtha feed bypasses the catalytic reformer.

FIG. 8 is a block flow diagram of a variation of the naphtha based steam cracking unit of FIG. 7, wherein a hydrocracker naphtha feed is supplied to the catalytic reformer with a portion of the heavy naphtha feed.

FIG. 9A is a block flow diagram of a naptha based ethylene plant wherein ethylene plant capacity is increased by increasing naphtha feed to the ethylene plant.

FIG. 9B is a block flow diagram of a naphtha based ethylene plant wherein ethylene plant capacity is increased by increasing naphtha feed to the ethylene plant and supplying a dearomatized heavy naphtha stream to the ethylene plant.

### DETAILED DESCRIPTION OF THE INVENTION

Detailed embodiments of the present invention are disclosed herein. However, it is understood that the disclosed embodiments are merely exemplary of the present invention, which can be embodied in various forms and are not to be construed as limitations of the invention. Specific structural, functional and process details disclosed herein are not intended to be limiting, but are merely illustrations that can be modified within the scope of the attached claims.

In addition to ethylene and propylene, naphtha based crackers can produce various by-products which can contain significant amounts of aromatics, such as for example, raw pyrolysis gasoline (C5 to 200° C.), which frequently can have an end boiling point similar to the naphtha feed. As steam crackers increase in size, dedicated facilities are typically integrated with the steam cracker to recover aromatic compounds. In recovering of aromatics from pyrolysis gasoline, the raw pyrolysis gasoline can be hydrogenated to saturate di-olefins and olefins, and the saturated pyrolysis gasoline can then be fed to an aromatics recovery unit.

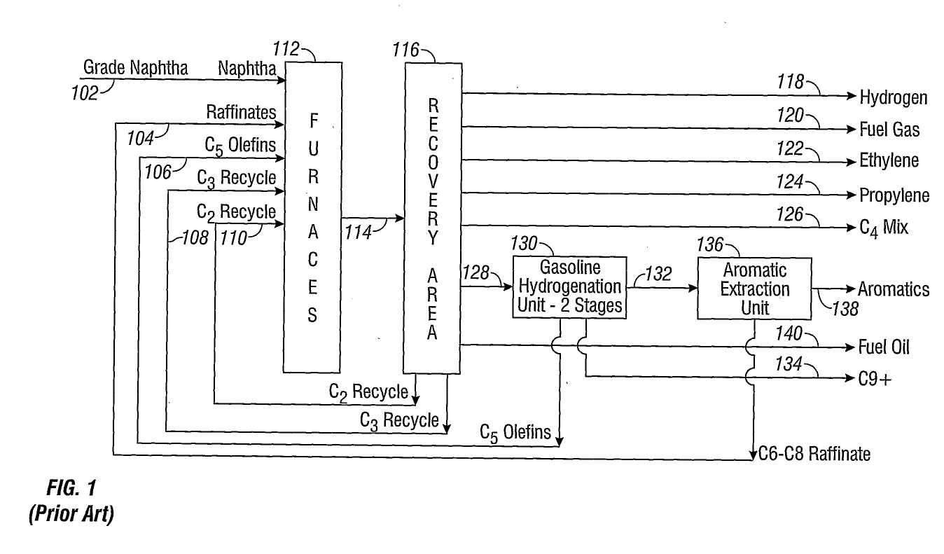

Referring to the drawings, wherein like referenced parts have like numerals, the design for a prior art naphtha based steam cracker with an aromatics extraction unit is shown in FIG. 1. The process can comprise a feed stream 102, a furnace 112, and a separation and recovery area 116. A feedstream of grade naphtha 102 is fed to furnaces 112 for cracking. Furnace effluent is supplied via line 114 to a recovery section 116, which can comprise various known means for the separation and recovery of mixed hydrocarbon streams, including but not limited to, fractionation, distillation, and the like. The separation and recovery process 116 can produce a variety of streams including hydrogen 118, fuel gas 120, ethylene 122, propylene 124, C4 mixed, and fuel oil 140 for recovery and export, and ethane 110 and propane 108 streams, which can be recycled to the furnace(s). An aromatics stream 128 can be recovered, and processed in a gasoline hydrogenation unit 130 producing a C5 olefin stream 106 which can be recycled to the furnace 112 as steam cracking feed, a C9+ fraction which can be recovered, and a hydrogenated C6-C8 stream 132, which can be supplied to an aromatics extraction unit 136. The aromatics extraction unit can produce an aromatics stream 140 for recovery and a C6-C8 raffinate stream 104, which can be recycled to the furnaces 112.

Preferably, the C6-C8 fraction of the furnace effluent can be hydrogenated in the two stage gasoline hydrogenation unit 130 to saturate diolefins in the first stage and to convert olefins to saturated compounds and remove of sulfur and nitrogen contaminants in the second stage. While single stage gasoline hydrogenation units are used in the art and can be used in the present invention, a two stage hydrogenation unit is preferable to achieve more complete removal of sulfur and nitrogen. Hydrogenation of the C6-C8 fraction is relatively expensive as removal of nitrogen is generally expensive. Hydrogenation of C6-C8 fraction is typically necessary to meet the sulfur specification requirements of aromatic products such as high purity benzene or nitration grade toluene. The second stage hydrogenated C6-C8 fraction supplied to the aromatics unit 136 can comprise benzene, toluene and C8 aromatics, as well as paraffins and naphthenes.

The aromatics extraction unit 136 can be provided to remove aromatics from the second stage hydrogenated C6-C8 fraction 132. The primary function of the aromatics extraction unit 136 is to separate aromatics, such as for example, benzene, toluene, and/or C8 aromatics, from the non-aromatic compounds which are identified as C6-C8 raffinate, typically comprising C6-C8 paraffins and naphthenes. For purposes of the specification and claims, raffinates comprise the portion of the feed which is not extracted and removed by the aromatics extraction unit, and may contain negligible undesired aromatics as well as hydrocarbons desired as feed to the cracking furnace 112. The C6-C8 raffinates can be recycled to the furnace for additional steam cracking. Separation of aromatics from non-aromatics can be achieved using conventional liquid-liquid extraction techniques and/or extractive distillation, as is known in the art. Commercially practiced technologies for the extraction of aromatics include: UOP-Sulfolane, UOP-Udex, UOP-Tetra, Uhde-Morphylex, IFP-DMSO, Lurgi-Arosolvan, Snamprogetti-FM, GTC-ED, and the like. Sample patents relating to the extraction of aromatics include U.S. Pat. Nos. 3,944,483; 5,310,480; 6,124,514; and 6,375,802; each of which is hereby incorporated herein by reference.

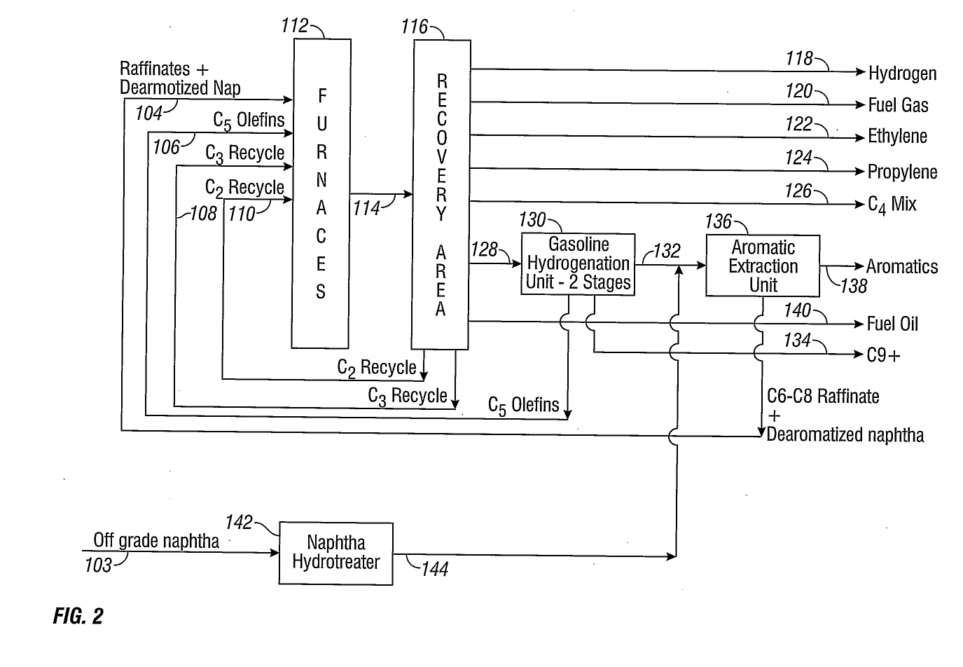

One embodiment of the present invention is shown in FIG. 2, wherein a naphtha dearomatization process can be integrated with the naphtha steam cracker for the upgrading of an off-grade naphtha feed for the production of light olefins. The process comprises a naphtha feed stream 103, a cracking furnace 112, and a separation and recovery area 116. A feed stream of naphtha 103 can be supplied to a naphtha hydrotreater 142, and fed to the aromatics extraction unit 136 via line 144. The aromatics extraction unit 136 can produce an aromatics stream 138 for collection, and a stream comprising dearomatized naphtha 104. The dearomatized naphtha stream 104 can be supplied to furnace 112 to produce a hydrocarbon effluent 114, which is supplied to separation and recovery area 116. The conventional separation and recovery area 116 can produce hydrogen 118, fuel gas 120, ethylene 122, propylene 126 and fuel oil 140 streams for collection. An ethane stream 110 and propane stream 108 can be separated from the furnace effluent 114 and recycled to the cracking furnace 112. A pyrolysis gasoline stream 128 can be supplied to a two stage gasoline hydrogenation unit 130, which produces a C5 fraction for recycle to the furnace 112, a C6-C8 fraction for supply to the aromatics extraction unit 136 via line 132, and a C9+ fraction for collection via line 134. The aromatics extraction unit 136 can produce an aromatics stream 138 for removal from the process and a C6-C8 raffinate stream 104, comprising C6-C8 paraffin and naphthenes, which can be combined with the dearomatized naphtha stream for supply to the furnace.

Naphtha dearomatization can include subjecting naphtha stream to hydrotreating in a naphtha hydrotreater unit to remove impurities. As shown in FIG. 2, the hydrotreated naphtha stream 144 can be mixed with a hydrogenated C6-C8 fraction 132 from the gasoline hydrogenation unit 130 and fed to the aromatics extraction unit 136. The aromatics extraction unit can produce a composite stream containing C6-C8 raffinate and dearomatized naphtha, and can be sent to the furnaces for steam cracking.

Depending on market conditions and availability, access to grade naphtha may not be sufficient to produce ethylene at maximum plant capacity. The shortfall in production can be made up by supplying off-grade naphtha as all or a portion of the required cracking feed, utilizing the present dearomatization process. Because of the lower price of off-grade naphtha, dearomatization and steam cracking of an off-grade feed can be highly profitable. Existing plants designed to operate with grade naphtha can be retrofitted to accommodate heavy naphtha feeds.

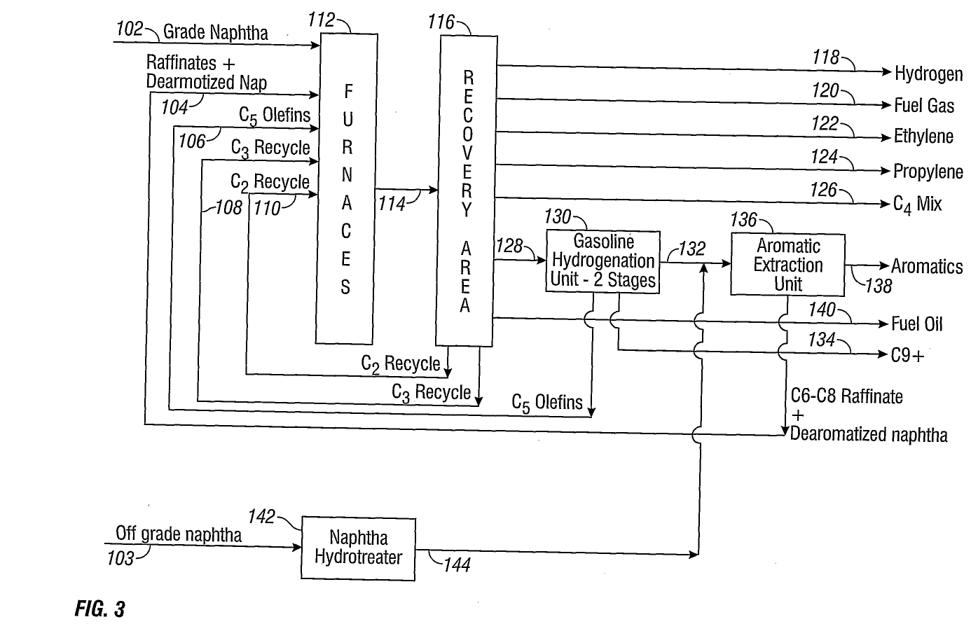

As shown in FIG. 3, an ethylene plant configuration for the combined use of grade and off-grade naphtha is similar to the ethylene plant of FIG. 2, with the addition of a naphtha feedstream supplied directly to the furnace. FIG. 3 shows a process for the production of olefins, wherein both grade and off-grade naphtha can be supplied as feed. The process can comprise a grade naphtha feed stream 102, an off-grade naphtha feed stream 103, a cracking furnace 112, and a separation and recovery area 116. A feedstream of grade naphtha 102 is fed to conventional furnaces 112 for cracking. Furnace effluent is supplied via line 114 to a recovery section 116, which can comprise various known means for the separation and recovery of mixed hydrocarbon streams. The separation and recovery process 116 can produce a variety of streams including hydrogen 118, fuel gas 120, ethylene 122, propylene 124, mixed C4, and fuel oil 140 for recovery and export, and can also produce ethane 110 and propane 108 streams, which can be recycled to the furnace 112 as a feedstream. A pyrolysis gasoline stream 128 can be recovered, and processed in two stage gasoline hydrogenation unit to produce a C5 olefin stream 106, which can be recycled to the furnace 112, a C9+ fraction which can be recovered, and a C6-C8 stream 132, which can be supplied to an aromatics extraction unit 136. The off-grade naphtha stream 103 can be supplied to naphtha hydrotreater 142, and then supplied via line 144 to the aromatics extraction unit 136, where it is combined with the C6-C8 stream 132. The aromatics extraction unit 136 produces an aromatics stream 138 for collection and a stream 104 comprising dearomatized naphtha and C6-C8 raffinate for supply to the furnace 112.

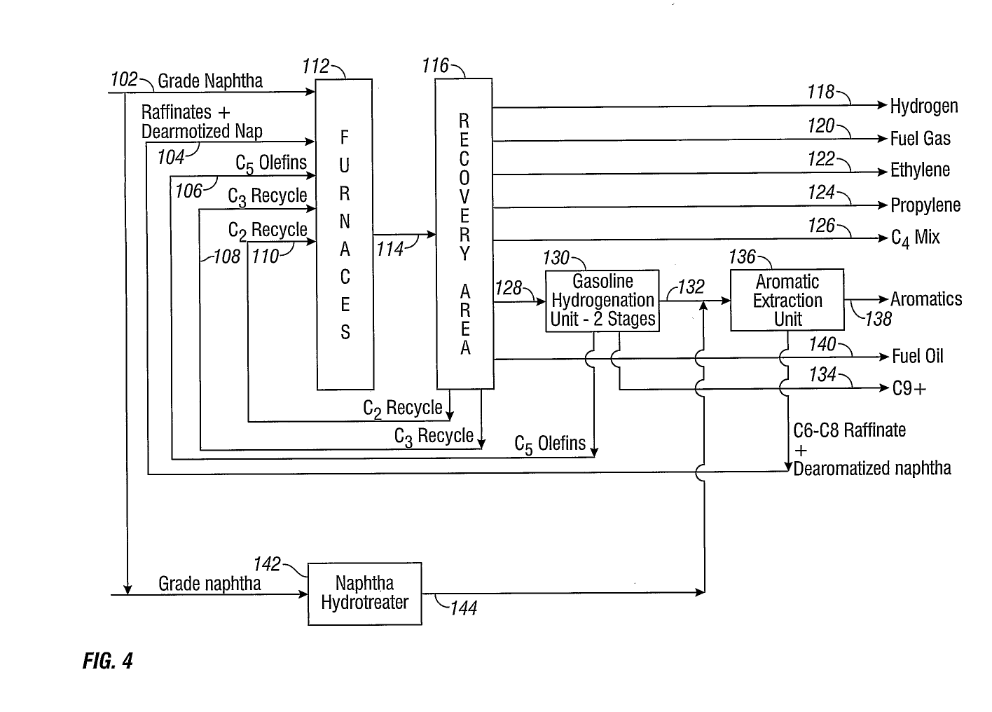

In a similar fashion, grade naphtha feed can be supplied to a hydrotreater and the aromatics extraction unit, as shown in FIG. 4, to increase the yield of the overall process. FIG. 4 shows a process with grade naphtha dearomatization to increase yield. The process can comprise a grade naphtha feed stream 102, a furnace 112, and a separation and recovery area 116. A feedstream of grade naphtha 102 is fed to furnaces 112 for cracking. A portion of the grade naphtha can be supplied to a naphtha hydrotreater 142 and aromatics extraction unit 136 prior to being fed to the furnace 112. Furnace effluent is supplied via line 114 to a recovery section 116, which can comprise various known means for the separation and recovery of mixed hydrocarbon streams. The separation and recovery process 116 can produce a variety of streams including hydrogen 118, fuel gas 120, ethylene 122, propylene 124, mixed C4, and fuel oil 140 streams for recovery, and can also produce ethane 110 and propane 108 streams, which can be recycled to the furnaces. A pyrolysis gasoline stream 128 can be recovered, and processed in a two stage gasoline hydrogenation unit to produce a C5 olefin stream 106 which can be recycled to the furnace 112, a C9+ fraction which can be recovered, and a C6-C8 stream 132 which can be supplied to an aromatics extraction unit 136 where it is combined with the hydrotreated naphtha stream 144. The aromatics extraction unit can produce an aromatics stream 140 for collection and a stream 104 comprising C6-C8 raffinate and dearomatized naphtha, which can be recycled to the furnaces 112 for cracking.

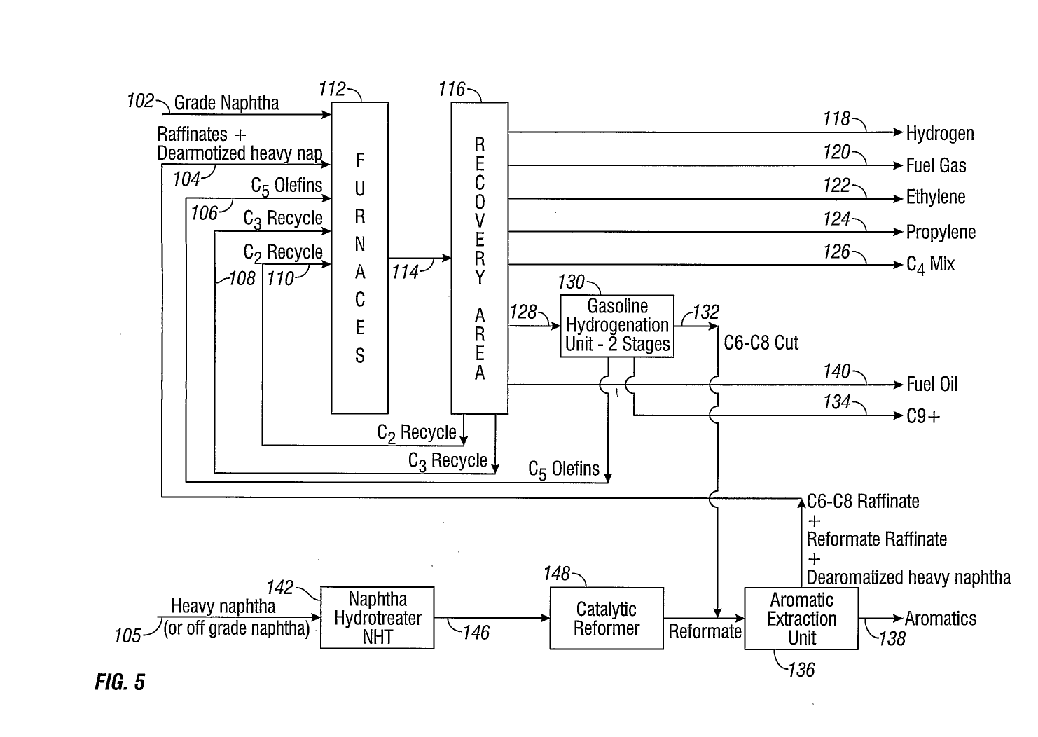

Integrating heavy naphtha dearomatization can have further advantages. In many petrochemical complexes, according to an embodiment of the invention, the steam cracker can be integrated with the aromatics production as shown in FIG. 5, where the aromatics extraction unit is common to both units. The process can comprise a feed stream 102, a furnace 112, and a separation and recovery unit 116. A feedstream of grade naphtha 102 can be fed to furnaces 112 for cracking to produce an effluent which can be supplied to the recovery and separation area 116 via line 114. The recovery and separation process can comprise a variety of known techniques, as described within this application, to produce a variety of streams including hydrogen 118, fuel gas 120, ethylene 122, propylene 124, a mixed C4 stream, and a fuel oil 140 for recovery. The recovery and separation can also recover streams of ethane 110 and propane 108 for recycle as feedstreams to the furnace. A pyrolysis gasoline stream can be supplied from the recovery and separation area 116 to a two stage gasoline hydrogenation unit 130 via line 128 to produce a C5 olefin fraction 106 that can also be recycled to the furnace 112 for further cracking. The second hydrogenation stage can produce a C6-C8 fraction 132, and a C9+ fraction 134. The C6-C8 fraction 132 can comprise aromatics, paraffins, and naphthenes, and can be supplied to an aromatics extraction unit 136. A feed stream of heavy or off-grade naphtha 105 can be supplied to a naphtha hydrotreater 142, and supplied via line 146 to catalytic reformer 148 to produce a reformate which can be supplied via line 149, where it is fed with the C6-C8 fraction 132 from the pyrolysis gasoline hydrogenation unit 130, to the aromatics extraction unit 136. The aromatics extraction unit 136 can produce an aromatics stream 138 for collection, and a furnace feed line 104 comprising C6-C8 raffinate, reformate raffinate, and dearomatized heavy naphtha.

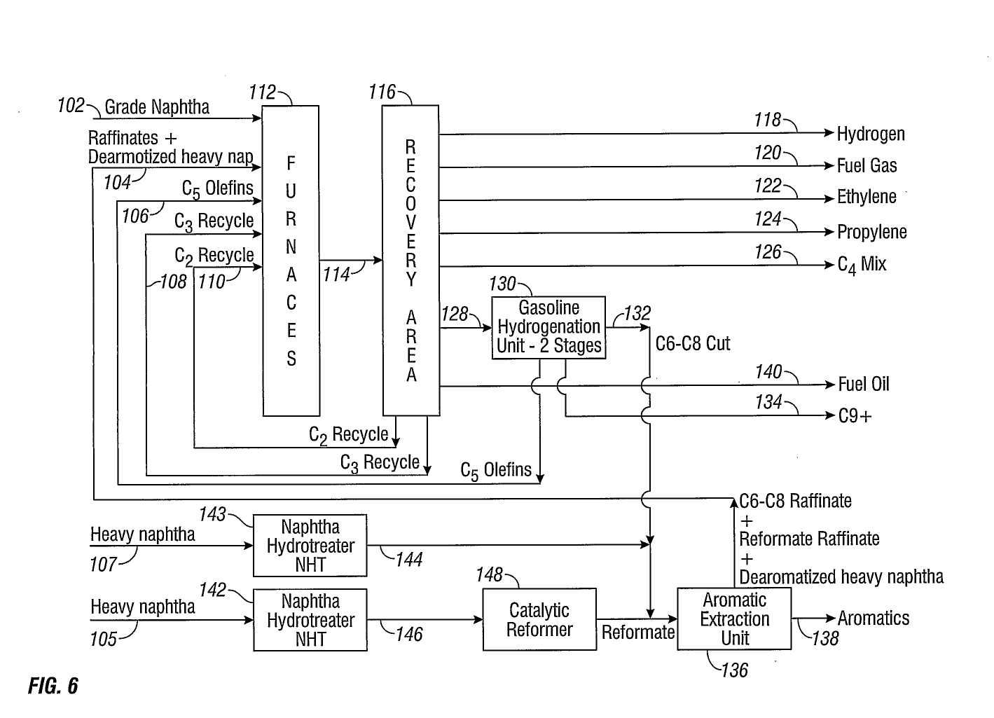

This arrangement of the invention can be easily adapted to integrate an additional heavy naphtha stream for dearomatization, followed by ethylene production, as shown in the FIG. 6, wherein a second heavy naphtha feedstream can be hydrotreated in a separate naphtha hydrotreater unit 143, and then fed to the aromatics extraction unit 136, along with the steam cracker C6-C8 fraction 132 and the reformate. The process can comprise a feed stream 102, a furnace 112, and a separation and recovery unit 116. A feedstream of grade naphtha 102 can be fed to furnaces 112 for cracking to produce an effluent which can be supplied to the recovery and separation area 116 via line 114. The recovery and separation process can comprise a variety of known techniques, as described within this application, to produce a variety of streams including hydrogen 118, fuel gas 120, ethylene 122, propylene 124, a mixed C4 stream, and a fuel oil 140 for recovery. The recovery and separation can also recover streams of ethane 110 and propane 108 for recycle to the furnaces. A pyrolysis gasoline stream can be supplied from the recovery and separation area 116 to a two stage gasoline hydrogenation unit 130 via line 128 to produce a C5 olefin fraction 106 that can be recycled to the furnace 112 for further cracking. The second hydrogenation stage can produce a C6-C8 fraction 132, and a C9+ fraction 134. The C6-C8 fraction 132 can be supplied to an aromatics extraction unit 136. A feed stream of heavy naphtha 105 can be supplied to a naphtha hydrotreater 142, and supplied via line 146 to catalytic reformer 148 to produce a reformate which can be supplied via line 149, where it is combined with the C6-C8 fraction 132 from the pyrolysis gasoline hydrogenation unit 130, to the aromatics extraction unit 136. A second heavy naphtha stream 107 can be supplied to a second naphtha hydrotreater 143, and via line 144 to be fed with the C6-C8 fraction 132 to the aromatics extraction unit 136. The heavy naphtha streams 105, 107 can be or include an off-grade naphtha, if desired. The aromatics extraction unit 136 produces an aromatics stream 138 for collection, and a stream in furnace feed line 104 comprising C6-C8 raffinate, reformate raffinate, and dearomatized heavy naphtha.

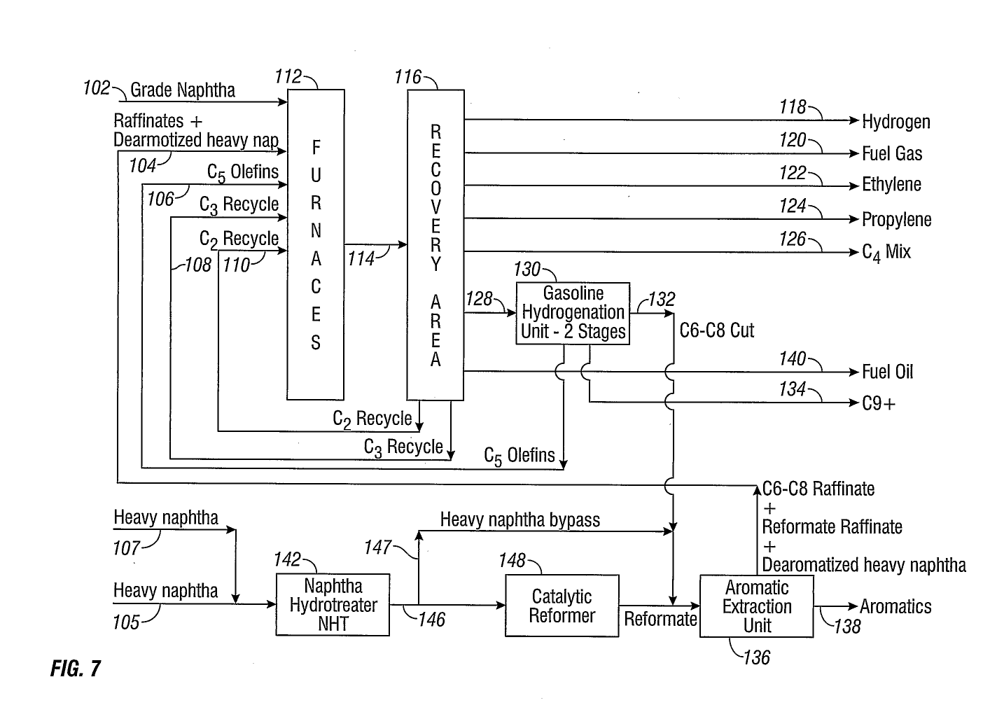

Further integration can be achieved by combining the two separate heavy naphtha streams 105, 107 from the FIG. 6 configuration, and supplying to a single hydrotreater 142, as shown in the FIG. 7, thereby reducing equipment and maintenance costs. As shown in FIG. 7, a portion of the hydrotreated heavy naphtha required for the steam cracker can bypass the catalytic reformer 148 for supply to the aromatics extraction unit 136. The process can comprise a feed stream 102, a furnace 112, and a separation and recovery unit 116. A feedstream of grade naphtha 102 can be fed to furnaces 112 for cracking to produce an effluent which is supplied to the recovery and separation area 116 via line 114. The recovery and separation process can comprise a variety of known techniques, as described within this application, to produce a variety of streams including hydrogen 118, fuel gas 120, ethylene 122, propylene 124, a mixed C4 stream, and a fuel oil 140 for recovery. The recovery and separation can also recover streams of ethane 110 and propane 108 for recycle to the furnaces. A pyrolysis gasoline stream 128 can be supplied from the recovery and separation area 116 to a two stage gasoline hydrogenation unit 130 to produce a C5 olefin fraction 106 that can be recycled to the furnace 112 for further cracking. The second hydrogenation stage can produce a C6-C8 fraction 132, and a C9+ fraction 134. The C6-C8 fraction 132 can comprise paraffins, naphthenes, and aromatics, and can be supplied to an aromatics extraction unit 136. A first feed stream of heavy naphtha 105 and a second stream of heavy naphtha 107 can be supplied to a naphtha hydrotreater 142. A first portion of the hydrotreater 142 effluent can be supplied via line 146 to catalytic reformer 148 to produce a reformate which can be supplied via line 149 to the aromatic extractions unit 136. A second portion of the hydrotreater effluent 142 can bypass the reformer 148 and can be supplied via line 147 to line 132 where it is combined with the C6-C8 fraction 132 from the pyrolysis gasoline hydrogenation unit 130, and then supplied to the aromatics extraction unit 136 where it is combined with the reformate. The heavy naphtha streams 105, 107 can alternatively be supplied with an off-grade naphtha is desired. The aromatics extraction unit 136 can produce an aromatics stream 138 for collection and a furnace feedstream 104 comprising C6-C8 raffinate, reformate raffinate, and dearomatized heavy naphtha.

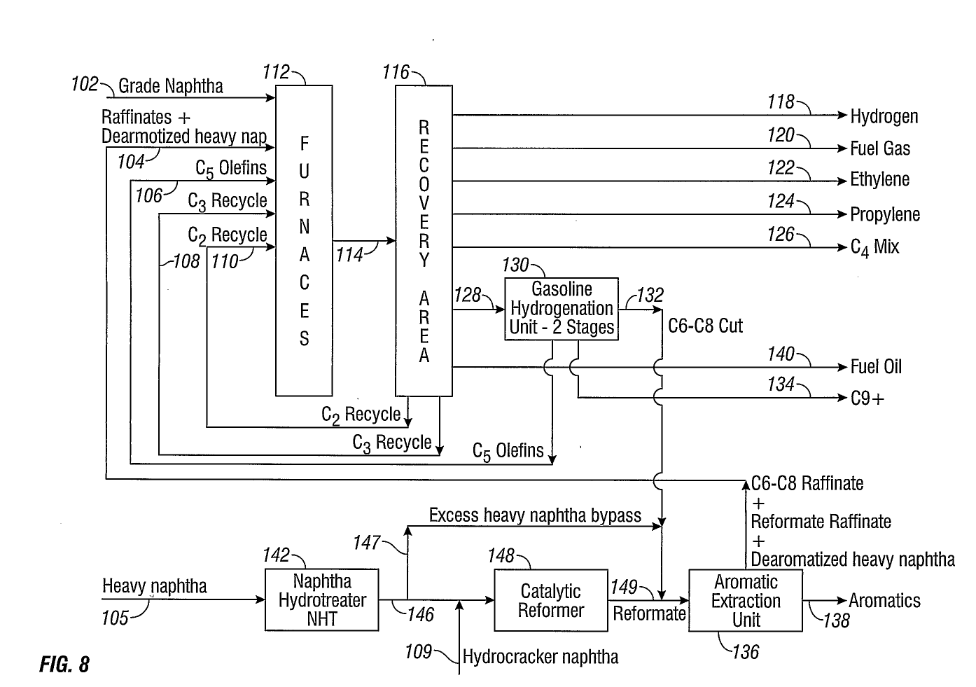

In some integrated ethylene-aromatics complexes, the catalytic reformers can process both heavy naphtha and hydrocracker naphtha, as shown in FIG. 8. Hydrocracker naphtha is usually of a higher quality than the heavy naphtha. The process can comprise a feed stream 102, a furnace 112, and a separation and recovery unit 116. A feedstream of grade naphtha 102 can be supplied to furnaces 112 for cracking to produce an effluent which is supplied to the recovery and separation area 116 via line 114. The recovery and separation process can comprise a variety of known techniques, as described within this application, to produce a variety of streams including hydrogen 118, fuel gas 120, ethylene 122, propylene 124, a mixed C4 stream, and fuel oil 140 for recovery. The recovery and separation can also recover streams of ethane 110 and propane 108 for recycle to the furnaces. A pyrolysis gasoline stream 128 can be supplied from the recovery and separation area 116 to a two stage gasoline hydrogenation unit 130 to produce a C5 olefin fraction 106 that can be recycled to the furnace 112 for further cracking. The second hydrogenation stage can produce a C6-C8 fraction 132 comprising paraffins, naphthenes and aromatics, and a C9+ fraction 134. The C6-C8 fraction 132 can be supplied to an aromatics extraction unit 136. A feed stream of heavy or off-grade naphtha 105 can be supplied to a naphtha hydrotreater 142, and all or a portion of the hydrotreated heavy naptha can be supplied via line 146 to catalytic reformer 148. All or a portion of the hydrotreated heavy naphtha can bypass the catalytic reformer 148 via line 147, and combine with the C6-C8 fraction 132 for supply to the aromatics extraction unit 136. The hydrotreater effluent can be combined with a hydrocracker naphtha feed stream 109 and supplied to the reformer 148 to produce a reformats. The reformate can be supplied via line 149, where it is combined with the C6-C8 fraction 132 from the pyrolysis gasoline hydrogenation unit 130 and the hydrotreated heavy naphtha 147, and supplied via 149 to the aromatics extraction unit 136. The aromatics extraction unit 136 can produce an aromatics stream 138 for collection, and a furnace feed line 104 comprising C6-C8 raffinate, reformate raffinate, and dearomatized heavy naphtha for cracking.

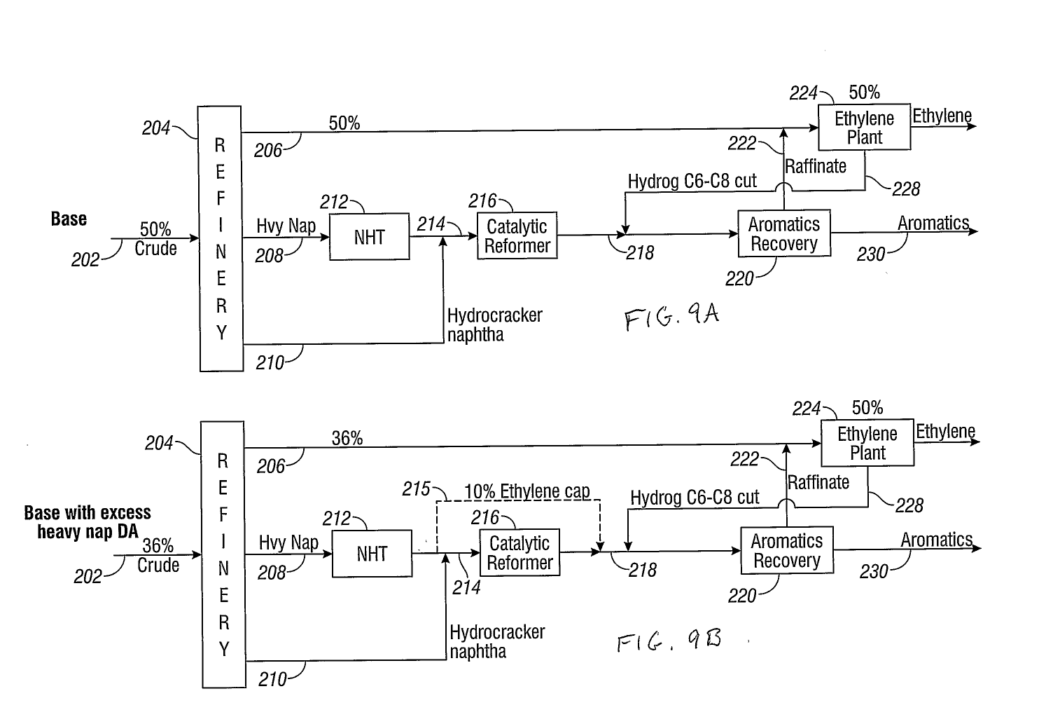

Dearomatization of the heavy naphtha can be useful in the case where the expansion of ethylene production in an integrated ethylene-aromatics complex is desired, as shown in FIGS. 9A and 9B. As shown in FIG. 9A, the base case ethylene plant comprises a refinery 204, an ethylene plant 224, and an aromatics extraction unit 220. Crude oil 202 is supplied to the refinery, producing streams of naphtha 206, heavy naphtha 208, and hydrocracker naphtha 210. Heavy naphtha 208 is supplied to naphtha hydrotreater 212 and the hydrotreated naphtha is supplied via line 210 to a catalytic reformer 216 where the hydrotreated naphtha is combined with hydrocracker naphtha supplied via 210. The reformer produces a reformate 218, which can be supplied to the aromatics recovery unit 220. The aromatics recovery unit 220 produces a raffinate stream 222 and an aromatics stream 230. The ethylene plant is supplied with grade naphtha 206 from the refinery and raffinate stream 222 to produce an ethylene product stream 226. The ethylene plant also produces a C6-C8 fraction 228 which can be hydrogenated (not shown) and supplied to combine with the reformate 218 in the aromatics recovery unit 220. To increase the ethylene capacity of the base case ethylene plant by 50%, crude feed to the refinery is increased by 50%.

Dearomatization of a heavy naphtha feed can provide an alternate means to increasing the ethylene capacity of an integrated ethylene-aromatics plant, as shown in FIG. 9B. The operation for the ethylene-aromatics plant shown in FIG. 9B is the same as that for the plant shown in FIG. 9A and described above. To increase ethylene capacity of the plant, a portion of the hydrotreated heavy naphtha from line 214 bypasses the catalytic reformer via line 215. The amount of hydrotreated heavy naphtha supplied to line 215 varies with the properties and composition of the heavy naphtha feed, but desirably can be calculated to account for a predetermined increase in the ethylene capacity of the plant, such as for example, a 10% increase in plant capacity. Reformate from reformer 220 and dearomatized heavy naphtha 215 are combined with a hydrogenated C6-C8 fraction 228 and supplied to the aromatics recovery unit 220, to produce an aromatics stream 230 and a mixed raffinate and dearomatized heavy naphtha stream 222 for supply to the ethylene plant 224. To increase total capacity by 10%, crude feed to the refinery is increased by 36%. Dearomatization of heavy naphtha can provide an increase in ethylene capacity of the plant by approximately 10%, thus providing a total increase in plant capacity of 150%. Excess heavy naphtha can be supplied to provide increased ethylene capacity of greater than 10%, and crude feed requirements can be reduced accordingly.

Naphtha feeds, whether grade or off-grade, often contain impurities which may present problems in ethylene production. Refineries typically utilize hydrotreating techniques to remove impurities present in the feedstock to protect catalytic reforming catalyst. Naphtha hydrotreaters are generally designed to produce hydrotreated naphtha streams having the following maximum allowable contaminant levels:

| Contaminant | Maximum level | |
|---|---|---|
| Sulfur | 1 | wt ppm |
| Nitrogen | 0.5 | wt ppm |
| Lead | 10 | wt ppb |
| Arsenic | 2 | wt ppb |
| Water | 10 | wt ppm |
| Chloride | 1 | wt ppm |

Use of current state of the art naphtha hydrotreaters may result in lower levels of contaminants and can include the removal of additional impurities. The contaminant levels noted above are generally considered acceptable for steam cracking feeds. Advantageously, the dearomatization process removes the bulk of the contaminants and provides a contaminant free dearomatized naphtha stream.

If the naphtha feed contains arsenic at a greater concentration than given above, the ethylene plant must include an arsenic removal system, such as for example, guard beds upstream from the hydrogenation units. By removing contaminants with the hydrotreater, the ethylene plant can be designed without separate contaminant removal systems, resulting in decreased construction and maintenance costs.

Because the dearomatized naphtha will have a low nitrogen content, the steam cracking byproduct raw pyrolysis gasoline will also have a low nitrogen content. The second stage hydrogenation of the raw pyrolysis gasoline may be less expensive due to a low severity design. In the case where 100% of the naphtha feed is supplied to both the hydrotreater and dearomatization units prior to cracking, the second stage of the gasoline hydrogenation unit may be designed for the removal of low level, such as for example at the part per billion (ppb) level, or for operation at low severity. In the case where a portion of the naphtha feed is supplied to both the hydrotreater and dearomatization units prior to cracking, lower severity nitrogen removal may be possible. However, while the dearomatized naphtha may have a lower sulfur content, sulfur may be added to facilitate steam cracking, and may require removal in the second stage of the gasoline hydrogenation unit.

Dearomatization of the naphtha can also reduce quench oil tower fouling. Polymers of styrene, indene and di-vinyl benzene are believed to contribute to quench oil tower fouling. Styrene is a product of the dehydrogenation of ethylbenzene. Indene can be produced by condensation reactions involving aromatic compounds. Divinyl benzene can be formed by the dehydrogenation of diethyl benzene. Polynuclear heavy aromatics formed by condensation reactions and present in the fuel oil streams can be responsible for fouling the bottom of the quench oil tower. Thus, removal of the aromatics from the steam cracker feed can reduce the formation of the aromatic compounds responsible for the quench oil tower fouling.

Reduction of quench oil tower fouling rates can result in longer run lengths and less frequent maintenance of the towers. Aromatics may still be formed due to the cracking reactions, but formation of compounds believed to cause fouling will be greatly reduced. Chemical additives designed to dissolve the polymers responsible for the quench oil tower fouling are known in the art and can be used in the present invention. However, the amount of chemical additive necessary can be reduced due to the dearomatization of the naphtha feed.

### EXAMPLES

A naphtha steam cracker according to the process configuration of FIG. 1 was modeled using open specification naphtha (hereinafter OSN or grade naphtha) as the feed for an ethylene plant having a capacity of 800 kTA (thousand metric tons per annum), at a severity corresponding to propylene to ethylene ratio of 0.5. Yields for OSN feed and the recycle streams were calculated using the Pycos model. In comparing the grade naphtha feed to off-grade feed streams, the naphtha steam cracker was modeled to first calculate overall material balance, and then to calculate total furnace effluents, which were used to characterize the size of major equipment for the ethylene plant. The calculations are compared against current equipment size requirements and capacities for existing ethylene plants to determine suitability of dearomatized and non-dearomatized off-grade naphtha.

Two off grade naphthas (hereinafter naphtha A and naphtha B) were selected to model the performance of a dearomatized off-grade naphtha feed. The composition of the off-grade naphtha feeds are shown in Table 1 below:

TABLE 1
Composition of Modeled Naphthas

| | OSN | Naphtha | Naphtha | Naphtha A | Naphtha B |
|---|---|---|---|---|---|
| | Naphtha | A | B | with DA | with DA |
| | Wt % | Wt % | Wt % | Wt % | Wt % |
| Normal Paraffins | 31.9 | 25.0 | 20.0 | 32.9 | 26.7 |
| Iso Paraffins | 34.4 | 30.0 | 21.0 | 39.5 | 28.0 |
| Naphthenes | 24.3 | 21.0 | 34.0 | 27.6 | 45.3 |
| Aromatics | 9.4 | 24.0 | 25.0 | 0.0 | 0.0 |
| Total | 100.0 | 100.0 | 100.0 | 100.0 | 100.0 |
| Specific gravity | 0.70 | 0.72 | 0.74 | 0.69 | 0.70 |
| Total Paraffins | 66.3 | 55.0 | 41.0 | 72.4 | 54.7 |
| Paraffins + | 90.6 | 76.0 | 75.0 | 100.0 | 100.0 |
| Naphthenes | | | | | |

As previously described, naphthas A and B are designated as off-grade because they do not meet the OSN minimum paraffins specifications and therefore are typically not used as steam cracker feeds. Table 1 also provides the compositions of dearomatized naphtha A and B streams.

By removing the aromatic compounds from the naphtha A feed, the paraffinic content can be increased to approximately 72% by weight, an increase in the paraffins content of approximately 31% over the non-dearomatized naphtha A feed. The dearomatized naphtha A feed meets the OSN specification and making the feed suitable for steam cracker feed. The paraffin content of the dearomatized naphtha B feed, having a paraffin content of approximately 55% by weight (an increase of approximately 34%), is still below 65% by weight, and would be rejected as a steam cracker feed.

### Example 1

In this example OSN based ethylene plant performance with OSN naphtha is compared to steam cracker performance with the naphtha A feed. The comparison shows that naphtha A is not well suited for processing in the OSN based steam cracker. Yields were calculated using the Pycos model. The naphtha A feed steam cracker performance is modeled to first calculate overall material balance, and then total furnace effluents are calculated to determine the size requirements for ethylene plant equipment. Comparisons of overall material balance, the major area sizes and fouling compounds in the quench oil tower feed of an OSN ethylene plant versus Naphtha A based ethylene plant are presented in Table 2.

TABLE 2
Steam Cracker Comparisons

| Case | 1 | 2 |
|---|---|---|
| | OSN | Naphtha |
| Naphtha | naphtha | A |
| Propylene/Ethylene ratio | 0.5 | 0.5 |
| Overall Material Balance | | |
| Feeds | kTA | kTA |
| JOS naphtha | 2,455.6 | — |
| Naphtha A or B | — | 2,850.5 |
| Total | 2,455.6 | 2,850.5 |
| Products | | |
| Hydrogen | 15.6 | 13.2 |
| FG | 389.7 | 367.4 |
| Ethylene | 800.0 | 800.0 |
| Propylene | 400.0 | 400.0 |
| Butadiene | 127.1 | 120.1 |
| Raffinate-1 | 126.9 | 121.3 |
| Benzene | 174.9 | 190.9 |
| Toluene | 105.0 | 212.4 |
| Xylenes+EB | 88.2 | 205.6 |
| C9+ Aromatics | 103.3 | 195.4 |
| FO | 125.0 | 224.2 |
| Total | 2,455.6 | 2,850.5 |
| Area Size factors | | |
| No of coils | 1.000 | 1.109 |
| Q, Fired, Total | 1.000 | 0.983 |
| QO tower size | 1.000 | 0.997 |
| FO Stripper | 1.000 | 1.962 |
| Distillate stripper | 1.000 | 1.344 |
| QW tower size | 1.000 | 0.966 |
| DS system | 1.000 | 1.135 |
| CG Compressor | 1.000 | 0.966 |
| C3-R Compressor | 1.000 | 1.006 |
| C2-R Compressor | 1.000 | 0.958 |
| Demethanizer size | 1.000 | 0.974 |
| Deethanizer size | 1.000 | 1.008 |
| Ethylene tower size | 1.000 | 1.005 |
| Depropanizer size | 1.000 | 0.996 |
| Propylene tower size | 1.000 | 0.998 |
| Debutanizer size | 1.000 | 0.975 |
| GHU-1st stage flow | 1.000 | 1.344 |
| Foulants in QOT feed | | |
| Styrene | 1.000 | 1.876 |
| Fuel oil | 1.000 | 1.962 |

Table 2 shows that a naphtha A feedstream, being paraffin poor and aromatics rich, produces approximately 79% more fuel oil than is produced by an OSN naphtha feed. The equipment size factor is greatest for the fuel oil stripper, meaning that the ethylene capacity for an OSN ethylene plant would be reduced by approximately 50% when supplied with a naphtha A feedstream, due to volume restrictions for the fuel oil stripper. Total capacity for a naphtha A feed ethylene plant is approximately 400 kTA (i.e. 50% of the ethylene capacity of the OSN ethylene plant).

### Example 2

An OSN feed ethylene plant is compared to a dearomatized naphtha A feed ethylene plantusing the similar calculations as used in Example 1. Table 3 shows comparisons of the overall material balance, the major equipment sizes, and fouling compounds in the quench oil tower feed for selected compounds in the furnace effluents.

As shown in Table 3, a dearomatized naphtha A feed, having had aromatics removed, produces approximately 65% of the fuel oil produced from an OSN naphtha feed. Similarly, the fuel oil stripper no longer limits the ethylene capacity of the plant. The largest equipment size factor for dearomatized naphtha A feed ethylene plant is 1.03 for the ethylene compressor, which implies that the naphtha feed plant will produce approximately 97% of the ethylene capacity of an OSN naphtha feed ethylene plant.

TABLE 3
Steam Cracker Comparisons

Case

| | 1 | 3 |
|---|---|---|
| Naphtha | OSN | Naphtha |
| | naphtha | A with DA |
| Propylene/Ethylene ratio | 0.5 | 0.5 |

Overall Material Balance

| Feeds | kTA | kTA |
|---|---|---|
| JOS naphtha | 2,455.6 | — |
| Naphtha A or B | — | 3,022.2 |
| Total | 2,455.6 | 3,022.2 |

Products

| | | |
|---|---|---|
| Hydrogen | 15.6 | 17.0 |
| FG | 389.7 | 405.7 |
| Ethylene | 800.0 | 800.0 |
| Propylene | 400.0 | 400.0 |
| Butadiene | 127.1 | 136.3 |
| Raffinate - 1 | 126.9 | 132.6 |
| Benzene | 174.9 | 221.3 |
| Toluene | 105.0 | 202.4 |
| Xylenes + EB | 88.2 | 295.7 |
| C9+ Aromatics | 103.3 | 329.7 |
| FO | 125.0 | 81.5 |
| Total | 2,455.6 | 3,022.2 |

Area Size factors

| | | |
|---|---|---|
| No of coils | 1.000 | 0.993 |
| Q, Fired, Total | 1.000 | 1.001 |
| QO tower size | 1.000 | 1.008 |
| FO Stripper | 1.000 | 0.598 |
| Distillate stripper | 1.000 | 0.839 |
| QW tower size | 1.000 | 1.023 |
| DS system | 1.000 | 0.945 |
| CG Compressor | 1.000 | 1.023 |
| C3-R Compressor | 1.000 | 0.997 |
| C2-R Compressor | 1.000 | 1.030 |
| Demethanizer size | 1.000 | 1.018 |
| Deethanizer size | 1.000 | 0.995 |
| Ethylene tower size | 1.000 | 0.997 |
| Depropanizer size | 1.000 | 1.005 |
| Propylene tower size | 1.000 | 1.003 |
| Debutanizer size | 1.000 | 1.029 |
| GHU - 1st stage flow | 1.000 | 0.839 |

Foulants in QOT feed

| | | |
|---|---|---|
| Styrene | 1.000 | 0.614 |
| Fuel oil | 1.000 | 0.598 |

The styrene and fuel oil content of the quench oil tower feed is indicative of quench oil tower fouling. In the case of a dearomatized naphtha A feedstream, styrene and fuel oil production is approximately 60% of that for an OSN feed, indicating that quench oil tower fouling should be substantially reduced when dearomatized naphtha A is supplied as the feed.

One consequence of dearomatizing the naphtha A feedstream is that on an overall basis, production of benzene and C8 aromatics can be maximized. A comparison of the overall material balances for benzene and C8 aromatics is shown below. On a fixed ethylene production basis, benzene production is 221 kTA for dearomatized naphtha A versus only 191 kTA for non-dearomatized naphtha A. Similarly C8 aromatics production on a fixed ethylene production basis is 296 kTA for dearomatized naphtha B versus 206 kTA for non-dearomatized naphtha B. On a fixed feed basis, benzene production for dearomatized naphtha A is 9% greater than for non-dearomatized naphtha A and C8 production for dearomatized naphtha A is 36% greater than for non-dearomatized naphtha A.

| | Fixed | Fixed | | |
|---|---|---|---|---|
| | Ethylene | feed | Naphtha A | |
| | Naphtha A | Naphtha A | with DA | % Increase |
| Naphtha Feed (kTA) | 2850.5 | 3022.2 | 3022.2 | |
| Benzene (kTA) | 190.9 | 202.3 | 221.3 | 9 |
| C8 Aromatics (kTA) | 205.6 | 218.0 | 295.7 | 36 |

### Example 3

In this example an OSN based steam cracker feed is compared to a naphtha B based steam cracker feed showing the performance and unsuitablity of naphtha B as a feed in an OSN based steam cracker. Yields for naphtha B feed and the recycle streams were calculated using the Pycos model. The naphtha B steam cracker is modeled to calculate overall material balance, and total furnace effluents, which are used to determine the size of major equipment for the ethylene plant. Table 4 shows a comparison of the overall material balance, the major equipment sizes, and fouling compounds in the quench oil tower feed for an OSN feed ethylene plant versus naphtha B feed based ethylene plant.

Table 4 shows that naphtha B, being paraffin poor and aromatics rich, produces more than twice the amount of fuel oil as compared with an OSN feed. The area size factor for the fuel oil stripper is 2.2, which implies that an OSN ethylene plant with a naphtha B feed will produce at 46% of the ethylene capacity of an OSN feed plant.

TABLE 4
Steam Cracker Comparisons

Case

| | 1 | 4 |
|---|---|---|
| Naphtha | OSN | Naphtha B |
| | naphtha | |
| Propylene/Ethylene ratio | 0.5 | 0.5 |

Overall Material Balance

| Feeds | kTA | kTA |
|---|---|---|
| JOS naphtha | 2,455.6 | — |
| Naphtha A or B | — | 2,903.7 |
| Total | 2,455.6 | 2,903.7 |

Products

| | | |
|---|---|---|
| Hydrogen | 15.6 | 12.1 |
| FG | 389.7 | 342.9 |
| Ethylene | 800.0 | 800.0 |
| Propylene | 400.0 | 400.0 |
| Butadiene | 127.1 | 144.9 |
| Raffinate-1 | 126.9 | 108.3 |
| Benzene | 174.9 | 189.6 |
| Toluene | 105.0 | 225.9 |
| Xylenes + EB | 88.2 | 219.1 |
| C9+ Aromatics | 103.3 | 219.7 |
| FO | 125.0 | 241.2 |
| Total | 2,455.6 | 2,903.7 |

Area Size factors

| | | |
|---|---|---|
| No of coils | 1.000 | 1.217 |
| Q, Fired, Total | 1.000 | 0.959 |
| QO tower size | 1.000 | 0.976 |
| FO Stripper | 1.000 | 2.159 |
| Distillate stripper | 1.000 | 1.393 |
| QW tower size | 1.000 | 0.939 |
| DS system | 1.000 | 1.154 |
| CG Compressor | 1.000 | 0.939 |
| C3-R Compressor | 1.000 | 0.997 |
| C2-R Compressor | 1.000 | 0.910 |
| Demethanizer size | 1.000 | 0.944 |
| Deethanizer size | 1.000 | 1.004 |
| Ethylene tower size | 1.000 | 1.002 |
| Depropanizer size | 1.000 | 1.003 |
| Propylene tower size | 1.000 | 1.005 |
| Debutanizer size | 1.000 | 1.000 |
| GHU - 1st stage flow | 1.000 | 1.393 |

Foulants in QOT feed

| | | |
|---|---|---|
| Styrene | 1.000 | 1.959 |
| Fuel oil | 1.000 | 2.159 |

### Example 4

In this example an OSN feed ethylene plant is compared with a dearomatized naphtha B ethylene plant feed using the same calculations. Table 5 shows the comparisons of overall material balance, the major equipment sizes, and fouling compounds in the quench oil tower feed for an OSN feed ethylene plant versus a dearomatized naphtha B feed ethylene plant.

TABLE 5
Steam Cracker Comparisons

Case

| | 1 | 5 |
|---|---|---|
| Naphtha | OSN | Naphtha |
| | naphtha | B with DA |
| Propylene/Ethylene ratio | 0.5 | 0.5 |

Overall Material Balance

| Feeds | kTA | kTA |
|---|---|---|
| JOS naphtha | 2,455.6 | — |
| Naphtha A or B | — | 3,273.5 |
| Total | 2,455.6 | 3,273.5 |

Products

| | | |
|---|---|---|
| Hydrogen | 15.6 | 17.4 |
| FG | 389.7 | 411.1 |
| Ethylene | 800.0 | 800.0 |
| Propylene | 400.0 | 400.0 |
| Butadiene | 127.1 | 151.5 |
| Raffinate-1 | 126.9 | 131.5 |
| Benzene | 174.9 | 283.6 |
| Toluene | 105.0 | 245.2 |
| Xylenes + EB | 88.2 | 342.9 |
| C9+ Aromatics | 103.3 | 388.2 |
| FO | 125.0 | 102.0 |
| Total | 2,455.6 | 3,273.5 |

Area Size factors

| | | |
|---|---|---|
| No of coils | 1.000 | 1.074 |
| Q, Fired, Total | 1.000 | 1.021 |
| QO tower size | 1.000 | 1.034 |
| FO Stripper | 1.000 | 0.794 |
| Distillate stripper | 1.000 | 1.049 |
| QW tower size | 1.000 | 1.033 |
| DS system | 1.000 | 1.010 |
| CG Compressor | 1.000 | 1.033 |
| C3-R Compressor | 1.000 | 0.992 |
| C2-R Compressor | 1.000 | 1.039 |
| Demethanizer size | 1.000 | 1.024 |
| Deethanizer size | 1.000 | 0.991 |
| Ethylene tower size | 1.000 | 0.994 |
| Depropanizer size | 1.000 | 1.007 |
| Propylene tower size | 1.000 | 1.002 |
| Debutanizer size | 1.000 | 1.056 |
| GHU - 1st stage flow | 1.000 | 1.049 |

Foulants in QOT feed

| | | |
|---|---|---|
| Styrene | 1.000 | 0.843 |
| Fuel oil | 1.000 | 0.794 |

Table 5 shows that a dearomatized naphtha B feed, having no aromatics, produces 80% of the fuel oil produced by the OSN feed. Similarly, the fuel oil stripper is no longer a limiting factor to ethylene capacity. The largest equipment size factor for a dearomatized naphtha B feed is 1.07 for the number of coils. Based upon this limitation, production for the dearomatized naphtha B feed is approximately 93% of the ethylene capacity of an OSN feed ethylene plant. Capacity for a dearomatized naphtha B feed is approximately twice that for the non-dearomatized naphtha B feed.

As previously noted, styrene and fuel oil content of quench oil tower feed can be indicative of quench oil tower fouling. For a dearomatized naphtha B feed, styrene and fuel oil production is approximately 85% of that for an OSN feed, indicating that quench oil tower fouling should be reduced with a dearomatized naphtha B feed.

On an overall basis, production of benzene and C8 aromatics is maximized when the dearomatized naphtha is used. Comparison of the naphtha B feed and dearomatized naphtha B feeds show that on fixed ethylene production basis, benzene production is 283.6 kTA for dearomatized naphtha B versus only 189.6 kTA for naphtha B. Similarly, C8 aromatics production on a fixed ethylene production is 342.9 kTA for dearomatized naphtha B versus 219.1 kTA for naphtha B. On a fixed feed basis, benzene production is increased with a dearomatized naphtha B feed to 284 kTA from 214 kTA, an increase of 33%. Similarly, C8 aromatics production is increased to 343 kTA from 247 kTA, an increase of 39% over an OSN feed. The calculations are shown below:

| | Fixed | Fixed | | |
|---|---|---|---|---|
| | Ethylene | Feed | Naphtha B | |
| | Naphtha B | Naphtha B | with DA | % Increase |
| Naphtha feed (kTA) | 2903.7 | 3273.5 | 3273.5 | |
| Benzene (kTA) | 189.6 | 213.7 | 283.6 | 33 |
| C8 Aromatics (kTA) | 219.1 | 247.0 | 342.9 | 39 |

### Example 5

An ethylene plant using the hybrid naphtha feed system presented in the FIG. 3 is shown here. The ethylene plant can be supplied with an 80% feed of grade OSN naphtha and a 20% feed of off grade naphtha A prepared using the dearomatization process of the present invention. Table 6 shows the comparisons of overall material balance, the major equipment sizes, and fouling compound compositions in the quench oil tower feed of an OSN feed versus a dearomatized naphtha B feed ethylene plant.

TABLE 6
Steam Cracker Comparisons

Case

| | 1 | 6 |
|---|---|---|
| Naphtha | OSN | 80/20 |
| | naphtha | OSN/A-DA |
| Propylene/Ethylene ratio | 0.5 | 0.5 |

Overall Material Balance

| Feeds | kTA | kTA |
|---|---|---|
| JOS naphtha | 2,455.6 | 1,964.5 |
| Naphtha A or B | — | 604.4 |
| Total | 2,455.6 | 2,568.9 |

Products

| | | |
|---|---|---|
| Hydrogen | 15.6 | 15.8 |
| FG | 389.7 | 392.9 |
| Ethylene | 800.0 | 800.0 |
| Propylene | 400.0 | 400.0 |
| Butadiene | 127.1 | 128.9 |
| Raffinate-1 | 126.9 | 128.0 |
| Benzene | 174.9 | 184.2 |
| Toluene | 105.0 | 124.5 |
| Xylenes + EB | 88.2 | 129.7 |
| C9+ Aromatics | 103.3 | 148.6 |
| FO | 125.0 | 116.3 |
| Total | 2,455.6 | 2,568.9 |

Area Size factors

| | | |
|---|---|---|
| No of coils | 1.000 | 0.999 |
| Q, Fired, Total | 1.000 | 1.000 |
| QO tower size | 1.000 | 1.002 |
| FO Stripper | 1.000 | 0.920 |
| Distillate stripper | 1.000 | 0.968 |
| QW tower size | 1.000 | 1.005 |
| DS system | 1.000 | 0.989 |
| CG Compressor | 1.000 | 1.005 |
| C3-R Compressor | 1.000 | 0.999 |
| C2-R Compressor | 1.000 | 1.006 |
| Demethanizer size | 1.000 | 1.004 |
| Deethanizer size | 1.000 | 0.999 |
| Ethylene tower size | 1.000 | 0.999 |
| Depropanizer size | 1.000 | 1.001 |
| Propylene tower size | 1.000 | 1.001 |
| Debutanizer size | 1.000 | 1.006 |
| GHU - 1st stage flow | 1.000 | 0.968 |

Foulants in QOT feed

| | | |
|---|---|---|
| Styrene | 1.000 | 0.923 |
| Fuel oil | 1.000 | 0.920 |

As shown in Table 6, all of the equipment size factors for the hybrid 80/20 case differ by less than 1%, indicating an ethylene production capacity using the hybrid naphtha feed of approximately 99%, as compared to the OSN feed ethylene plant production. A shortage of grade quality naphtha feedstock meeting OSN specifications can be mitigated using off grade naphtha by employing this invention. Levels of styrene and fuel oil in the quench oil tower for the hybrid naphtha feed can be reduced by approximately 8%, as compared to the OSN naphtha feed.

### Example 6

The benefits of a dearomatized naphtha feed for the production at a propylene to ethylene ratio of 0.45 is shown in Table 7, where comparisons of overall material balance, the major equipment sizes, and fouling compounds in the quench oil tower feed for an OSN feed ethylene plant and a dearomatized heavy naphtha A feed ethylene plant are given. Dearomatized naphtha A, having no aromatic compounds present, produces approximately 63% of the fuel oil produced by OSN feed, and the fuel oil stripper is no longer a limiting factor to ethylene capacity. The largest area size factor for a dearomatized naphtha A feed is 1.03 for the ethylene compressor, corresponding to an ethylene production capacity of approximately 97%, as compared to capacity of an OSN naphtha feed.

TABLE 7
Steam Cracker Comparisons

Case

| | Base | 1 |
|---|---|---|
| Naphtha | No DeArom | w DeArom |
| | JOS | A |
| Propylene/Ethylene ratio | 0.45 | 0.45 |

Overall Material Balance

| Feeds | kTA | kTA |
|---|---|---|
| JOS naphtha | 2,388.4 | — |
| Naphtha A or B | — | 2,949.4 |
| Total | 2,388.4 | 2,949.4 |

Products

| | | |
|---|---|---|
| Hydrogen | 16.9 | 18.1 |
| FG | 396.9 | 414.7 |
| Ethylene | 800.0 | 800.0 |
| Propylene | 360.0 | 360.0 |
| Butadiene | 119.2 | 127.6 |
| Raffinate-1 | 102.6 | 109.1 |
| Benzene | 181.2 | 231.7 |
| Toluene | 101.7 | 196.8 |
| Xylenes + EB | 83.8 | 286.3 |
| C9+ Aromatics | 97.9 | 318.9 |
| FO | 128.2 | 86.3 |
| Total | 2,388.4 | 2,949.4 |

Area Size Factors

| | | |
|---|---|---|
| No of coils | 1.000 | 0.963 |
| Q, Fired, Total | 1.000 | 1.002 |
| QO tower size | 1.000 | 1.011 |
| FO stripper | 1.000 | 0.628 |
| Distillate stripper | 1.000 | 0.843 |
| QW tower size | 1.000 | 1.025 |
| DS system | 1.000 | 0.948 |
| CG Compressor | 1.000 | 1.025 |
| C3-R Compressor | 1.000 | 0.998 |
| C2-R Compressor | 1.000 | 1.033 |
| Demethanizer size | 1.000 | 1.020 |
| Deethanizer size | 1.000 | 0.996 |
| Ethylene tower size | 1.000 | 0.998 |
| Depropanizer size | 1.000 | 1.005 |
| Propylene tower size | 1.000 | 1.003 |
| Debutanizer size | 1.000 | 1.033 |
| GHU - 1st stage flow | 1.000 | 0.843 |

Foulants in QOT feed

| | | |
|---|---|---|
| Styrene | 1.000 | 0.659 |
| Fuel oil | 1.000 | 0.628 |

Styrene and fuel oil content of the quench oil tower feed can be a good indicator of fouling. For a dearomatized naphtha A feed, styrene and fuel oil production can be reduced to approximately 66% of that of OSN feed ethylene plant, indicating that quench oil tower fouling rates should be reduced when using a dearomatized naphtha A feed stream as compared to an OSN feed stream.

### Heavy Naphtha Feed

To assess the suitability of dearomatized heavy naphtha we selected two heavy naphthas, hereinafter heavy naphthas A and B, as shown in the Table 8 below. As shown in Table 9, a naphtha steam cracker has been modeled according to the process design configuration of FIG. 5, using OSN as the feed for an ethylene capacity of 800 kTA at a severity corresponding to a propylene to ethylene ratio of 0.50. Yields for the OSN and the recycle streams were calculated using the Pycos model. The naphtha steam cracker was modeled to first calculate the overall material balance, and then to determine total furnace effluents which can be used to characterize the size of major equipment for the ethylene plant.

TABLE 8
List of study naphthas

| | | Heavy | Heavy | Heavy | Heavy |
|---|---|---|---|---|---|
| | OSN | Naphtha | Naphtha | Naphtha A | Naphtha B |
| | Naphtha | A | B | with DA | with DA |
| | Wt % | Wt % | Wt % | Wt % | Wt % |
| Normal Paraffins | 31.9 | 25.0 | 20.0 | 32.9 | 26.7 |
| Iso Paraffins | 34.4 | 30.0 | 21.0 | 39.5 | 28.0 |
| Naphthenes | 24.3 | 21.0 | 34.0 | 27.6 | 45.3 |
| Aromatics | 9.4 | 24.0 | 25.0 | 0.0 | 0.0 |
| Total | 100.0 | 100.0 | 100.0 | 100.0 | 100.0 |
| Specific gravity | 0.70 | 0.74 | 0.76 | 0.71 | 0.73 |
| Total Paraffins | 66.3 | 55.0 | 41.0 | 72.4 | 54.7 |
| Paraffins + | 90.6 | 76.0 | 75.0 | 100.0 | 100.0 |
| Naphthenes | | | | | |

TABLE 9
Steam Cracker Comparisons

Case

| | 1 | 2 |
|---|---|---|
| Naphtha | OSN | Heavy Nap |
| | naphtha | A |
| Propylene/Ethylene ratio | 0.5 | 0.5 |

Overall Material Balance

| Feeds | kTA | kTA |
|---|---|---|
| JOS naphtha | 2,455.6 | — |
| Naphtha A or B | — | 2,850.1 |
| Total | 2,455.6 | 2,850.1 |

Products

| | | |
|---|---|---|
| Hydrogen | 15.6 | 13.3 |
| FG | 389.7 | 367.3 |
| Ethylene | 800.0 | 800.0 |
| Propylene | 400.0 | 400.0 |
| Butadiene | 127.1 | 120.1 |
| Raffinate-1 | 126.9 | 121.1 |
| Benzene | 174.9 | 190.7 |
| Toluene | 105.0 | 212.3 |
| Xylenes + EB | 88.2 | 205.5 |
| C9+ Aromatics | 103.3 | 195.3 |
| FO | 125.0 | 224.5 |
| Total | 2,455.6 | 2,850.1 |

Area Size factors

| | | |
|---|---|---|
| No of coils | 1.000 | 1.080 |
| Q, Fired, Total | 1.000 | 0.981 |
| QO tower size | 1.000 | 0.997 |
| FO Stripper | 1.000 | 1.966 |
| Distillate stripper | 1.000 | 1.343 |
| QW tower size | 1.000 | 0.965 |
| DS system | 1.000 | 1.135 |
| CG Compressor | 1.000 | 0.965 |
| C3-R Compressor | 1.000 | 1.006 |
| C2-R Compressor | 1.000 | 0.957 |
| Demethanizer size | 1.000 | 0.974 |
| Deethanizer size | 1.000 | 1.008 |
| Ethylene tower size | 1.000 | 1.005 |
| Depropanizer size | 1.000 | 0.996 |
| Propylene tower size | 1.000 | 0.998 |
| Debutanizer size | 1.000 | 0.974 |
| GHU - 1st stage flow | 1.000 | 1.343 |

Foulants in QOT feed

| | | |
|---|---|---|
| Styrene | 1.000 | 1.877 |
| Fuel oil | 1.000 | 1.966 |

Note that heavy naphthas A and B have low paraffin contents and therefore would normally be rejected as steam cracker feeds. Table 8 shows the paraffin and aromatics compositions of dearomatized heavy naphtha A and B feeds. By removing aromatics from heavy naphtha A, the paraffinic content can be increased to approximately 72% by weight, an increase of approximately 31% over the non-dearomatized heavy naphtha A paraffinic content, which would be acceptable for use as a steam cracker feed. The dearomatized heavy naphtha B paraffinic content is approximately 55%, an increase of approximately 34% over the non-dearomatized heavy naphtha B paraffinic content, but still below acceptable levels. Therefore, dearomatized heavy naphtha B would likely be rejected as a steam cracker feed.

### Example 7

In this example, grade naphtha steam cracker performance is compared to heavy naphtha A based steam cracker performance to demonstrate the lack of suitability of a heavy naphtha A feedstream for ethylene production. Yields were calculated using the Pycos model. The heavy naphtha A feed steam cracker is modeled to first calculate the overall material balance, and then total furnace effluents are used to determine the size of major equipment for the ethylene plant. Table 9 shows the comparisons of overall material balance, the major equipment sizes and fouling compounds in the quench oil tower feed of an OSN ethylene plant versus heavy naphtha A based ethylene plant.

Table 9 shows that heavy naphtha A, paraffin poor and aromatics rich, produces approximately 96% more fuel oil than an OSN feedstream. The equipment size factor for the fuel oil stripper is the largest, meaning that to process heavy naphtha A, the ethylene capacity of an OSN ethylene plant would be reduced by approximately 50%, due to the reduced capacity of the fuel oil stripper. Thus, the heavy naphtha A feedstream can produce approximately 400 kTA ethylene when using a heavy naphtha A feedstock in an OSN ethylene plant.

### Example 8

An OSN naphtha feedstream ethylene plant is compared with a dearomatized heavy naphtha A feedstream ethylene plant. Table 10 shows a comparison of the overall material balance, the major equipment sizes, fouling compounds present in the quench oil tower feed, and selected products from the furnace effluents of ethylene plant for the OSN naphtha feed plant versus a dearomatized heavy naphtha A feedstream ethylene plant.

Table 10 shows that the dearomatized heavy naphtha A feedstream, having no aromatics present, produces only 60% of the fuel oil produced by a grade naphtha feedstream ethylene plant. The fuel oil stripper is no longer a limiting factor to the ethylene plant capacity. The largest equipment size factor for using dearomatized heavy naphtha A feedstream is 1.03 for the ethylene compressor, implying that the dearomatized heavy naphtha A feedstream ethylene plant will produce approximately 97% of the ethylene capacity obtainable with an OSN naphtha feedstream. In addition, a dearomatized naphtha A feedstream can produce more than twice the ethylene able to be produced with a non-dearomatized naphtha feedstream.

TABLE 10
Steam Cracker Comparisons

Case

| | 1 | 3 |
|---|---|---|
| Naphtha | OSN | Heavy Nap |
| | naphtha | A with DA |
| Propylene/Ethylene ratio | 0.5 | 0.5 |

Overall Material Balance

| Feeds | kTA | kTA |
|---|---|---|
| JOS naphtha | 2,455.6 | — |
| Naphtha A or B | — | 3,020.6 |
| Total | 2,455.6 | 3,020.6 |

Products

| | | |
|---|---|---|
| Hydrogen | 15.6 | 16.9 |
| FG | 389.7 | 405.1 |
| Ethylene | 800.0 | 800.0 |
| Propylene | 400.0 | 400.0 |
| Butadiene | 127.1 | 136.3 |
| Raffinate-1 | 126.9 | 132.5 |
| Benzene | 174.9 | 221.0 |
| Toluene | 105.0 | 202.3 |
| Xylenes + EB | 88.2 | 295.5 |
| C9+ Aromatics | 103.3 | 329.5 |
| FO | 125.0 | 81.4 |
| Total | 2,455.6 | 3,020.6 |

Area Size factors

| | | |
|---|---|---|
| No of coils | 1.000 | 0.957 |
| Q, Fired, Total | 1.000 | 1.001 |
| QO tower size | 1.000 | 1.007 |
| FO Stripper | 1.000 | 0.597 |
| Distillate stripper | 1.000 | 0.839 |
| QW tower size | 1.000 | 1.023 |
| DS system | 1.000 | 0.944 |
| CG Compressor | 1.000 | 1.023 |
| C3-R Compressor | 1.000 | 0.997 |
| C2-R Compressor | 1.000 | 1.029 |
| Demethanizer size | 1.000 | 1.018 |
| Deethanizer size | 1.000 | 0.995 |
| Ethylene tower size | 1.000 | 0.997 |
| Depropanizer size | 1.000 | 1.005 |
| Propylene tower size | 1.000 | 1.003 |
| Debutanizer size | 1.000 | 1.029 |
| GHU - 1st stage flow | 1.000 | 0.839 |

Foulants in QOT feed

| | | |
|---|---|---|
| Styrene | 1.000 | 0.614 |
| Fuel oil | 1.000 | 0.597 |

Styrene and fuel oil content of the quench oil tower feed can be indicative of the likelihood quench oil tower fouling. For a dearomatized heavy naphtha A feedstream, styrene and fuel oil production is approximately 60% of that of an OSN naphtha feed, indicating that quench oil tower fouling should be reduced when a dearomatized heavy naphtha A feedstream is steam cracked in the ethylene plant.

One result of dearomatizing the heavy naphtha feedstream is that on an overall basis, production of benzene and C8 aromatics can be maximized. A comparison of the overall material balances from Tables 9 and 10 shows that on a fixed ethylene production basis, benzene production is approximately 221 kTA for dearomatized heavy naphtha A, versus approximately 191 kTA for naphtha A. Similarly, C8 aromatics production for the fixed ethylene production case is 296 kTA for the dearomatized heavy naphtha A feed versus 206 kTA for naphtha A feed. On a fixed feed basis, benzene production for the dearomatized heavy naphtha A is 221 kTA versus 202 kTA for the non-dearomatized heavy naphtha A, an increase of approximately 9%. Similarly, C8 aromatics production for dearomatized heavy naphtha A is 296 kTA, compared with 218 kTA for the heavy naphtha A, an increase of approximately 36%.

| | Fixed | Fixed | | |
|---|---|---|---|---|
| | Ethylene | Feed | DA | |
| | Heavy | Heavy | Heavy | |
| | Naphtha A | Naphtha A | Naphtha A | % Increase |
| Naphtha feed (kTA) | 2850 | 3021 | 3021 | |
| Benzene (kTA) | 191 | 202 | 221 | 9 |
| C8 Aromatics (kTA) | 206 | 218 | 296 | 36 |

Thus, with a fixed feed, steam cracking of dearomatized heavy naphtha A shows an increase in both benzene and C8 aromatics production compared to the steam cracking of heavy naphtha A.

### Example 9

In this example, an OSN feedstream based ethylene plant is compared to a heavy naphtha B feed to show the suitability of the heavy naphtha B feedstream for processing. Yields for heavy naphtha B feed and the recycle streams were calculated using the Pycos model. The heavy naphtha B steam cracker is modeled to first calculate overall material balances, and then total furnace effluents are used to calculate the size of major equipment for the ethylene plant. The Table 11 provides comparisons of overall material balances, the major equipment sizes, and fouling compounds present in the quench oil tower feed of an OSN naphtha ethylene plant versus the heavy naphtha B based ethylene plant.

TABLE 11
Steam Cracker Comparisons

Case

| | 1 | 4 |
|---|---|---|
| Naphtha | OSN | Heavy Nap |
| | naphtha | B |
| Propylene/Ethylene ratio | 0.5 | 0.5 |

Overall Material Balance

| Feeds | kTA | kTA |
|---|---|---|
| JOS naphtha | 2,455.6 | — |
| Naphtha A or B | — | 2,902.4 |
| Total | 2,455.6 | 2,902.4 |

Products

| | | |
|---|---|---|
| Hydrogen | 15.6 | 12.1 |
| FG | 389.7 | 342.5 |
| Ethylene | 800.0 | 800.0 |
| Propylene | 400.0 | 400.0 |
| Butadiene | 127.1 | 145.1 |
| Raffinate-1 | 126.9 | 108.2 |
| Benzene | 174.9 | 189.6 |
| Toluene | 105.0 | 225.5 |
| Xylenes + EB | 88.2 | 219.0 |
| C9+ Aromatics | 103.3 | 219.4 |
| FO | 125.0 | 241.0 |
| Total | 2,455.6 | 2,902.4 |

Area Size factors

| | | |
|---|---|---|
| No of coils | 1.000 | 1.190 |
| Q, Fired, Total | 1.000 | 0.960 |
| QO tower size | 1.000 | 0.976 |
| FO Stripper | 1.000 | 2.156 |
| Distillate stripper | 1.000 | 1.392 |
| QW tower size | 1.000 | 0.938 |
| DS system | 1.000 | 1.154 |
| CG Compressor | 1.000 | 0.938 |
| C3-R Compressor | 1.000 | 0.997 |
| C2-R Compressor | 1.000 | 0.909 |
| Demethanizer size | 1.000 | 0.944 |
| Deethanizer size | 1.000 | 1.004 |
| Ethylene tower size | 1.000 | 1.002 |
| Depropanizer size | 1.000 | 1.003 |
| Propylene tower size | 1.000 | 1.005 |
| Debutanizer size | 1.000 | 1.000 |
| GHU - 1st stage flow | 1.000 | 1.392 |

Foulants in QOT feed

| | | |
|---|---|---|
| Styrene | 1.000 | 1.959 |
| Fuel oil | 1.000 | 2.156 |

Table 11 provides a comparison showing that heavy naphtha B feed, being paraffin poor and aromatics rich, can produces more than twice the amount fuel oil as is produced by a grade naphtha process. The equipment size factor for the fuel oil stripper is 2.16 and implies that heavy naphtha B feed ethylene plant will produce approximately 46% of ethylene capacity compared to an OSN naphtha feed.

### Example 10

An OSN naphtha feed ethylene plant is compared to a dearomatized heavy naphtha B feed. Table 12 presents the comparisons of overall material balances, the major equipment sizes, and fouling compounds present in the quench oil tower feed for the OSN naphtha ethylene plant versus the dearomatized heavy naphtha B feed ethylene plant.

TABLE 12
Steam Cracker Comparisons

Case

| | 1 | 5 |
|---|---|---|
| Naphtha | OSN | Heavy Nap |
| | naphtha | B with DA |
| Propylene/Ethylene ratio | 0.5 | 0.5 |

Overall Material Balance

| Feeds | kTA | kTA |
|---|---|---|
| JOS naphtha | 2,455.6 | — |
| Naphtha A or B | — | 3,272.0 |
| Total | 2,455.6 | 3,272.0 |

Products

| | | |
|---|---|---|
| Hydrogen | 15.6 | 17.4 |
| FG | 389.7 | 410.6 |
| Ethylene | 800.0 | 800.0 |
| Propylene | 400.0 | 400.0 |
| Butadiene | 127.1 | 151.5 |
| Raffinate-1 | 126.9 | 131.5 |
| Benzene | 174.9 | 283.3 |
| Toluene | 105.0 | 245.0 |
| Xylenes + EB | 88.2 | 342.8 |
| C9+ Aromatics | 103.3 | 387.7 |
| FO | 125.0 | 102.2 |
| Total | 2,455.6 | 3,272.0 |

Area Size factors

| | | |
|---|---|---|
| No of coils | 1.000 | 1.041 |
| Q, Fired, Total | 1.000 | 1.025 |
| QO tower size | 1.000 | 1.034 |
| FO Stripper | 1.000 | 0.796 |
| Distillate stripper | 1.000 | 1.049 |
| QW tower size | 1.000 | 1.032 |
| DS system | 1.000 | 1.010 |
| CG Compressor | 1.000 | 1.032 |
| C3-R Compressor | 1.000 | 0.992 |
| C2-R Compressor | 1.000 | 1.038 |
| Demethanizer size | 1.000 | 1.023 |
| Deethanizer size | 1.000 | 0.991 |
| Ethylene tower size | 1.000 | 0.994 |
| Depropanizer size | 1.000 | 1.007 |
| Propylene tower size | 1.000 | 1.002 |
| Debutanizer size | 1.000 | 1.056 |
| GHU - 1st stage flow | 1.000 | 1.049 |

Foulants in QOT feed

| | | |
|---|---|---|
| Styrene | 1.000 | 0.843 |
| Fuel oil | 1.000 | 0.796 |

Table 12 shows that dearomatized heavy naphtha B, having no aromatics present, produces approximately 80% of the fuel oil produced by an OSN naphtha feed, meaning the fuel oil stripper is no longer a limiting factor to ethylene capacity. The largest equipment size factor for the dearomatized heavy naphtha B feed is 1.05 for the GHU feed, which implies that the ethylene plant will be able to produce at approximately 95% of ethylene capacity with a dearomatized heavy naphtha B feed, as compared to approximately 46% using non-dearomatized heavy naphtha B feed.

Styrene and fuel oil production is approximately 85% of that of for a grade naphtha feed, indicating that quench oil tower fouling should be reduced for a dearomatized heavy naphtha B feedstream.

A comparison of Tables 11 and 12 shows that on fixed ethylene production basis, benzene production is 283 kTA for dearomatized heavy naphtha B feedstream versus 190 kTA for a naphtha B feedstream. Similarly, C8 aromatics production for a fixed ethylene production is 343 kTA for dearomatized heavy naphtha B feedstream versus 219 kTA for heavy naphtha B feedstream. On a fixed feed basis, benzene production for the dearomatized heavy naphtha B is 283 kTA, compared with 214 kTA for the heavy naphtha B feed, an increase of approximately 33%. Similarly, C8 aromatics production for the dearomatized heavy naphtha B is 343 kTA, compared with 247 kTA for the heavy naphtha B, an increase of approximately 39%.

| | Fixed | Fixed | | |
|---|---|---|---|---|
| | Ethylene | Feed | Heavy | |
| | Heavy | Heavy | Naphtha B | |
| | Naphtha B | Naphtha B | with DA | % Increase |
| Naphtha feed (kTA) | 2902 | 3272 | 3272 | |
| Benzene (kTA) | 190 | 214 | 283 | 33 |
| C8 Aromatics (kTA) | 219 | 247 | 343 | 39 |

### Example 11

A mixed feed system utilizing a hybrid feed comprising 80% OSN naphtha and 20% dearomatized heavy naphtha A, as shown in FIG. 13, is provided here. Table 13 shows a comparison of overall material balances, the major equipment sizes, and fouling compounds present in the quench oil tower feed for an OSN naphtha feed ethylene plant versus a dearomatized heavy naphtha B feed ethylene plant. A comparison of the equipment sizes is given in Table 13 and shows that for all of the equipment size factors for the mixed 80/20 case differ by less than 1%, indicating that in using the hybrid feed an ethylene capacity of approximately 99% can be obtained, compared to the OSN naphtha feedstock.

TABLE 13
Steam Cracker Comparisons

Case

| | 1 | 6 |
|---|---|---|
| Naphtha | OSN | 80/20 |
| | naphtha | OSN/A-DA |
| Propylene/Ethylene ratio | 0.5 | 0.5 |

Overall Material Balance

| Feeds | kTA | kTA |
|---|---|---|
| JOS naphtha | 2,455.6 | 1,964.5 |
| Naphtha A or B | — | 604.1 |
| Total | 2,455.6 | 2,568.6 |

Products

| | | |
|---|---|---|
| Hydrogen | 15.6 | 15.8 |
| FG | 389.7 | 392.8 |
| Ethylene | 800.0 | 800.0 |
| Propylene | 400.0 | 400.0 |
| Butadiene | 127.1 | 128.9 |
| Raffinate-1 | 126.9 | 128.0 |
| Benzene | 174.9 | 184.2 |
| Toluene | 105.0 | 124.5 |
| Xylenes + EB | 88.2 | 129.6 |
| C9+ Aromatics | 103.3 | 148.5 |
| FO | 125.0 | 116.3 |
| Total | 2,455.6 | 2,568.6 |

Area Size factors

| | | |
|---|---|---|
| No of coils | 1.000 | 0.991 |
| Q, Fired, Total | 1.000 | 1.000 |
| QO tower size | 1.000 | 1.001 |
| FO Stripper | 1.000 | 0.919 |
| Distillate stripper | 1.000 | 0.968 |
| QW tower size | 1.000 | 1.005 |
| DS system | 1.000 | 0.989 |
| CG Compressor | 1.000 | 1.005 |
| C3-R Compressor | 1.000 | 0.999 |
| C2-R Compressor | 1.000 | 1.006 |
| Demethanizer size | 1.000 | 1.004 |
| Deethanizer size | 1.000 | 0.999 |
| Ethylene tower size | 1.000 | 0.999 |
| Depropanizer size | 1.000 | 1.001 |
| Propylene tower size | 1.000 | 1.001 |
| Debutanizer size | 1.000 | 1.006 |
| GHU - 1st stage flow | 1.000 | 0.968 |

Foulants in QOT feed

| | | |
|---|---|---|
| Styrene | 1.000 | 0.923 |
| Fuel oil | 1.000 | 0.919 |

By using the mixed grade and dearomatized heavy naphtha feed, a shortage of quality grade naphtha can be mitigated without a decrease in ethylene production. Concentration of styrene and fuel oil in the quench oil tower feed can be decreased by approximately 8%, which can reduce quench oil tower fouling.

### Example 12

Table 14 shows the advantages for dearomatizing the heavy naphtha A feedstream for the production of ethylene at a propylene:ethylene ratio of 0.45. Table 14 shows comparisons of overall material balance, the major equipment sizes, and fouling compounds present in the quench oil tower feed feed of grade naphtha feed ethylene plant versus a dearomatized heavy naphtha A feed ethylene plant operating at a P/E ratio of 0.45.

TABLE 14
Steam Cracker Comparisons

Case

| | Base | 1 |
|---|---|---|
| Naphtha | OSN | Heavy Nap |
| | naphtha | A with DA |
| Propylene/Ethylene ratio | 0.45 | 0.45 |

Overall Material Balance

| Feeds | kTA | kTA |
|---|---|---|
| JOS naphtha | 2,388.4 | — |
| Naphtha A or B | — | 2,949.3 |
| Total | 2,388.4 | 2,949.3 |

Products

| | | |
|---|---|---|
| Hydrogen | 16.9 | 18.1 |
| FG | 396.9 | 414.4 |
| Ethylene | 800.0 | 800.5 |
| Propylene | 360.0 | 360.2 |
| Butadiene | 119.2 | 127.8 |
| Raffinate-1 | 102.6 | 109.0 |
| Benzene | 181.2 | 231.5 |
| Toluene | 101.7 | 196.7 |
| Xylenes + EB | 83.8 | 286.2 |
| C9+ Aromatics | 97.9 | 318.8 |
| FO | 128.2 | 86.1 |
| Total | 2,388.4 | 2,949.2 |

Area Size factors

| | | |
|---|---|---|
| No of coils | 1.000 | 0.928 |
| Q, Fired, Total | 1.000 | 1.003 |
| QO tower size | 1.000 | 1.011 |
| Distillate stripper | 1.000 | 0.842 |
| QW tower size | 1.000 | 1.025 |
| DS system | 1.000 | 0.948 |
| CG Compressor | 1.000 | 1.025 |
| CG MW | 1.000 | 0.989 |
| C3-R Compressor | 1.000 | 0.998 |
| C2-R Compressor | 1.000 | 1.032 |
| Demethanizer size | 1.000 | 1.020 |
| Deethanizer size | 1.000 | 0.996 |
| Ethylene tower size | 1.000 | 0.998 |
| Depropanizer size | 1.000 | 1.005 |
| Propylene tower size | 1.000 | 1.003 |
| Debutanizer size | 1.000 | 1.034 |
| GHU - 1st stage flow | 1.000 | 0.842 |

Foulants in QOT feed

| | | |
|---|---|---|
| Styrene | 1.000 | 0.656 |
| Fuel oil | 1.000 | 0.626 |

Table 14 comparison shows that dearomatized heavy naphtha A, having no aromatics present, produces approximately 63% of the fuel oil produced from an OSN naphtha feedstock, and that the fuel oil stripper is no longer a limiting factor to the ethylene capacity. The largest equipment size factor for the dearomatized heavy Naphtha A feed is 1.03 for the ethylene compressor, implying that the ethylene plant will be able to operate at 97% ethylene capacity using dearomatized heavy naphtha A feed as compared to using OSN naphtha feedstock.

For a dearomatized heavy naphtha A feed, of the present example, styrene and fuel oil production can be approximately 63% of that of the OSN naphtha feed, indicating that quench oil tower fouling should be reduced when dearomatized heavy naphtha A is used in the cracking process instead of the grade naphtha.

Numerous embodiments and alternatives thereof have been disclosed. While the above disclosure includes the best mode belief in carrying out the invention as contemplated by the inventors, not all possible alternatives have been disclosed. For that reason, the scope and limitation of the present invention is not to be restricted to the above disclosure, but is instead to be defined and construed by the appended claims.

What is claimed:

1. An olefins process for steam cracking an aromatics-containing naphtha stream comprising:
recovering olefins and pyrolysis gasoline streams from a steam cracking furnace effluent;
hydrogenating the pyrolysis gasoline stream and recovering a C6-C8 stream therefrom;
hydrotreating an aromatics-containing naphtha stream to obtain a naphtha feed stream lean in nitrogen, sulfur, arsenic, lead, or a combination thereof;
dearomatizing the C6-C8 stream with the naphtha feed stream in a common aromatics extraction unit to obtain a raffinate stream; and
feeding the raffinate stream to the steam cracking furnace.

2. The process of claim 1 wherein the aromatics-containing naphtha stream comprises a paraffins content of less than 65 weight percent.

3. The process of claim 1 wherein the aromatics-containing naphtha stream comprises an aromatics content of 10 weight percent or more.

4. The process of claim 1 wherein the steam cracking furnace effluent comprises a propylene to ethylene weight ratio from 0.3 to 0.8 (same cracker severity as spec naphtha feed).

5. The process of claim 1 further comprising feeding a second naphtha stream to the steam cracking furnace, wherein the second naphtha stream comprises 65 weight percent or more paraffins and no more than 10 weight percent aromatics.

6. The process of claim 1 wherein the pyrolysis gasoline is hydrogenated at less severe operating conditions.

7. The process of claim 1 wherein fouling in a quench oil tower receiving the steam furnace cracking effluent is inhibited.

8. The process of claim 1 further comprising recovering ethane and propane from the steam cracking furnace effluent and recycling the recovered ethane and propane to the steam cracking furnace.

9. The process of claim 1 further comprising recovering a C5 olefins stream from the pyrolysis gasoline hydrogenation and recycling the C5 olefins stream to the steam cracking furnace.

10. The process of claim 1 further comprising hydrotreating a second naphtha stream, wherein the second naphtha stream comprises 65 weight percent or more paraffins and no greater than 10 weight percent aromatics.

11. The process of claim 1 wherein the aromatics containing naphtha stream comprises heavy naphtha.

12. An olefins process for steam cracking a naphtha stream comprising aromatics, the process comprising:
recovering olefins and pyrolysis gasoline streams from a steam cracking furnace effluent;
hydrogenating the pyrolysis gasoline stream and recovering a C6-C8 stream therefrom;
hydrotreating a heavy naphtha stream comprising aromatics to obtain a heavy naphtha stream lean in nitrogen, sulfur, arsenic, lead, or a combination thereof;
reforming the hydrotreated heavy naphtha stream in a catalytic reformer to obtain a reformate comprising aromatics;
dearomatizing the C6-C8 stream with the reformate in a common aromatics unit to obtain a mixed stream comprising C6-C8 raffinate, reformate raffinate, and a dearomatized heavy naphtha stream; and
feeding the mixed stream to the steam cracking furnace.

13. The process of claim 12, further comprising hydrotreating a second aromatics-containing heavy naphtha stream in a second hydrotreater to obtain a second hydrotreated heavy naphtha stream lean in nitrogen, sulfur, arsenic, lead, or a combination thereof; and dearomatizing the heavy naphtha stream, the reformate and the C6-C8 stream in the common aromatics extraction unit.

14. The process of claim 12, further comprising:
supplying a portion of the hydrotreated heavy naphtha stream to the reformer; and
dearomatizing the hydrotreated heavy naphtha stream with the C6-C8 stream and the reformate raffinate.

15. The process of claim 14, further comprising reforming a hydrocracker naphtha stream with a portion of the hydrotreated heavy naphtha in the catalytic reformer to obtain a reformate stream.

16. An olefins process unit for steam cracking an aromatics-containing naphtha stream comprising:
one or more steam cracking furnaces to produce a pyrolysis effluent;
a recovery unit to recover olefins and pyrolysis gasoline streams from the pyrolysis effluent;
a gasoline hydrogenation unit to hydrogenate the pyrolysis gasoline stream and recover a C6-C8 stream;
a hydrotreating unit to remove nitrogen, sulfur, arsenic, lead, or a combination thereof from an aromatics-containing naphtha stream to obtain a naphtha feed stream;
a common aromatics extraction unit to dearomatize the C6-C8 stream together with the naphtha feed stream to obtain a raffinate stream; and
a line to feed the raffinate stream to the steam cracking furnace.

17. The olefins process unit of claim 16 further comprising lines to recycle ethane and propane streams from the recovery unit to the steam cracking furnace.

18. The olefins process unit of claim 16 further comprising a line to recycle a C5 olefins stream from the gasoline hydrogenation unit to the steam cracking furnace.
